SEQUENCE LISTING (12 sequences)

SEQ ID NO: 1 - DNA, 25 bp, unidentified
  source 1..25: mol_type = other DNA
gcgcacatgaggatcacccatgtgc

SEQ ID NO: 2 - protein, 117 aa, unidentified
MASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIY

SEQ ID NO: 3 - DNA, 26 bp, unidentified
  source 1..26: mol_type = other DNA
ataaggagtttatatggaaaccctta

SEQ ID NO: 4 - protein, 128 aa, unidentified
MSKTIVLSVGEATRTLTEIQSTADRQIFEEKVGPLVGRLRLTASLRQNGAKTAYRVNLKLDQADVVDCSTSVCGELPKVRYTQVWSHDVTIVANSTEASRKSLYDLTKSLVATSQVEDLVVNLVPLGR

SEQ ID NO: 5 - DNA, 19 bp, unidentified
  source 1..19: mol_type = other DNA
ctgaatgcctgcgagcatc

SEQ ID NO: 6 - protein, 62 aa, unidentified
MKSIRCKNCNKLLFKADSFDHIEIRCPRCKRHIIMLNACEHPTEKHCGKREKITHSDETVRY

SEQ ID NO: 7 - DNA, 66 bp, unidentified
  source 1..66: mol_type = other DNA
ttcttgtcgtacttatagatcgctacgttatttcaattttgaaaatctgagtcctgggagtgcgga

SEQ ID NO: 8 - protein, 609 aa, unidentified
MSGWESYYKTEGDEEAEEEQEENLEASGDYKYSGRDSLIFLVDASKAMFESQSEDELTPFDMSIQCIQSVYISKIISSDRDLLAVVFYGTEKDKNSVNFKNIYVLQELDNPGAKRILELDQFKGQQGQKRFQDMMGHGSDYSLSEVLWVCANLFSDVQFKMSHKRIMLFTNEDNPHGNDSAKASRARTKAGDLRDTGIFLDLMHLKKPGGFDISLFYRDIISIAEDEDLRVHFEESSKLEDLLRKVRAKETRKRALSRLKLKLNKDIVISVGIYNLVQKALKPPPIKLYRETNEPVKTKTRTFNTSTGGLLLPSDTKRSQIYGSRQIILEKEETEELKRFDDPGLMLMGFKPLVLLKKHHYLRPSLFVYPEESLVIGSSTLFSALLIKCLEKEVAALCRYTPRRNIPPYFVALVPQEEELDDQKIQVTPPGFQLVFLPFADDKRKMPFTEKIMATPEQVGKMKAIVEKLRFTYRSDSFENPVLQQHFRNLEALALDLMEPEQAVDLTLPKVEAMNKRLGSLVDEFKELVYPPDYNPEGKVTKRKHDNEGSGSKRPKVEYSEEELKTHISKGTLGKFTVPMLKEACRAYGLKSGLKKQELLEALTKHFQD

SEQ ID NO: 9 - protein, 485 aa, unidentified
MVRSGNKAAVVLCMDVGFTMSNSIPGIESPFEQAKKVITMFVQRQVFAENKDEIALVLFGTDGTDNPLSGGDQYQNITVHRHLMLPDFDLLEDIESKIQPGSQQADFLDALIVSMDVIQHETIGKKFEKRHIEIFTDLSSRFSKSQLDIIIHSLKKCDISERHSIEWPCRLTIGSNLSIRIAAYKSILQERVKKTWTVVDAKTLKKEDIQKETVYCLNDDDETEVLKEDIIQGFRYGSDIVPFSKVDEEQMKYKSEGKCFSVLGFCKSSQVQRRFFMGNQVLKVFAARDDEAAAVALSSLIHALDDLDMVAIVRYAYDKRANPQVGVAFPHIKHNYECLVYVQLPFMEDLRQYMFSSLKNSKKYAPTEAQLNAVDALIDSMSLAKKDEKTDTLEDLFPTTKIPNPRFQRLFQCLLHRALHPREPLPPIQQHIWNMLNPPAEVTTKSQIPLSKIKTLFPLIEAKKKDQVTAQEIFQDNHEDGPTAK

SEQ ID NO: 10 - DNA, 10 bp, unidentified
  source 1..10: mol_type = other DNA
aatttttgga

SEQ ID NO: 11 - protein, 83 aa, unidentified
GSVIDVSSQRVNVQRPLDALGNSLNSPVIIKLKGDREFRGVLKSFDLHMNLVLNDAEELEDGEVTRRLGTVLIRGDNIVYISP

SEQ ID NO: 12 - DNA, 32 bp, unidentified
  source 1..32: mol_type = other DNA
wsbwsdwshwsvwsbwswshwsvwsbwsdwsh

(19) United States
(12) Patent Application Publication
(10) Pub. No.: US 2026/0071269 A1
(43) Pub. Date: Mar. 12, 2026

(54) TRAFFICKED RNAS FOR ASSESSMENT OF CELL-CELL CONNECTIVITY AND NEUROANATOMY

(71) Applicants: THE BROAD INSTITUTE, INC., Cambridge, MA (US); THE GENERAL HOSPITAL CORPORATION, Boston, MA (US); PRESIDENT AND FELLOWS OF HARVARD COLLEGE, Cambridge, MA (US)

(72) Inventors: Michael John DOLAN, Cambridge, MA (US); Alex BUCKLEY, Cambridge, MA (US); Judy LUU, Cambridge, MA (US); Michael KIM, Cambridge, MA (US); Evan MACOSKO, Boston, MA (US)

(73) Assignees: THE BROAD INSTITUTE, INC., Cambridge, MA (US); THE GENERAL HOSPITAL CORPORATION, Boston, MA (US); PRESIDENT AND FELLOWS OF HARVARD COLLEGE, Cambridge, MA (US)

(21) Appl. No.: 19/235,474

(22) Filed: Jun. 11, 2025

Related U.S. Application Data
(63) Continuation of application No. PCT/US23/83537, filed on Dec. 12, 2023.
(60) Provisional application No. 63/387,020, filed on Dec. 12, 2022.

(51) Int. Cl. C12Q 1/6874; C07K 14/47; C12N 15/86; C12Q 1/6806; C12Q 1/6855; G01N 33/68

(52) U.S. Cl. CPC C12Q 1/6874; C07K 14/4702; C12N 15/86; C12Q 1/6806; C12Q 1/6855; G01N 33/6845; C07K 2319/85; C12N 2750/14143

(57) ABSTRACT

The present disclosure relates to compositions and methods for tracking and spatially localizing a cell-expressed fusion protein within the cell (with the fusion protein optionally associated with a subcellular compartment, organelle, synapse, or the like), in a manner that minimizes any disruptive impact upon the cell, at least until the detection process is initiated. Use of transcriptomics and/or barcode nucleic acid detection is employed to assess both spatial localization of intracellularly tagged fusion proteins and to establish cell-cell connectivity, e.g., in neurons across a synapse, by associating axonal identities with individual neurons at the molecular tag and transcriptome level.

### CROSS-REFERENCE TO RELATED APPLICATION

This application is a continuation under 35 U.S.C. § 111 (a) of International Application No. PCT/US2023/083537, filed Dec. 12, 2023, which claims the benefit of U.S. Provisional Application No. 63/387,020, filed Dec. 12, 2022, entitled “TRAFFICKED RNAS FOR ASSESSMENT OF CELL-CELL CONNECTIVITY AND NEUROANATOMY.” The entire contents of the aforementioned applications are incorporated herein by reference.

### GOVERNMENT SUPPORT CLAUSE

This invention was made with government support under Grant Nos. AG058488, HG010647, and MH130468 awarded by the National Institutes of Health. The government has certain rights in the invention.

### SEQUENCE LISTING

The instant application contains a Sequence Listing which has been filed electronically in extensible Markup Language (XML) format and is hereby incorporated by reference in its entirety. Said XML copy, created on Dec. 12, 2023, is named BN00007 1542_SequenceListing.xml and is 13,008 bytes in size.

### FIELD OF THE INVENTION

The invention relates generally to methods and compositions for detection of trafficked proteins and associated RNAs in a cell, tissue or organism.

### BACKGROUND OF THE INVENTION

Synapses define the points of communication between neurons, controlling the flow of information across neural circuits. Changes in number of synapses or neuronal projections occur in many diseases of the brain. However, no technology exists to link synaptic or neuroanatomical information with single cell gene expression, which could be used to infer neuronal identity. Moreover, current approaches to determining synaptic neuroanatomy are laborious and low throughput.

In the mammalian brain, highly heterogeneous neuronal cell types make specific synaptic connections with stereotyped partners during development. Together, these connectivity patterns define neuronal circuits, which have diverse, specialized roles in controlling brain function. Through the use of genetically defined tools for modulating neuronal circuits and synaptic connections, research has begun to reveal how particular cell types and circuits participate in brain function. Decades of prior research have identified changes in connectivity associated with nearly every major brain disease, often occurring early in pathogenesis and with potentially causal roles. Even with such progress, a need exists for enhanced detection of neuronal connectivity at scale in neuronal systems, particularly where perturbation of the neuronal system by the detection system is minimized.

### BRIEF SUMMARY OF THE INVENTION

The instant disclosure is based, at least in part, upon discovery of a system capable of simultaneously acquiring neuroanatomical and gene expression information from single neurons. The system disclosed herein specifically combines the following features to provide spatially-localized sequencing readouts that are capable of providing molecular characterization of individual neuronal synapse regions: the first feature is molecular technology that is capable of efficiently transporting mRNA to intracellular compartments, while the second feature is creation of modifications to single-cell and spatial transcriptomics workflows to enable accurate readout of trafficked barcodes alongside transcriptional information within a cell (e.g., a neuronal cell).

In certain aspects, the instant disclosure provides a system that is capable of measuring connectivity of cells/neurons, in a manner that maintains the health of such cells/neurons throughout introduction of exogenous plasmids/nucleic acids and tagging/trafficking/localization steps, until detection steps are performed.

In one aspect, the instant disclosure provides a composition for tagging the localization of a fusion protein, and/or a fusion protein-associated vesicle, synapse and/or organelle in a cell, tissue or organism, the composition including: a) a first plasmid capable of being expressed in a cell, where the first plasmid encodes for the fusion protein, where the fusion protein includes a first domain including (encoding for) a vesicle-, synapse- and/or organelle-associated protein or a polypeptide sequence that binds a vesicle-, synapse- and/or organelle-associated protein and a second domain including a selective nucleic acid binding protein; and b) (i) a second plasmid capable of being expressed in a cell, where the second plasmid encodes for an oligoribonucleotide including a selective protein binding nucleic acid domain, and a barcode nucleic acid or other nucleic acid, where the selective protein binding nucleic acid domain is capable of binding the selective nucleic acid binding protein encoded for by the first plasmid; or (ii) an oligonucleotide including a selective protein binding nucleic acid domain, and a barcode nucleic acid or other nucleic acid, where the selective protein binding nucleic acid domain is capable of binding the selective nucleic acid binding protein encoded for by the first plasmid.

In one embodiment, the selective nucleic acid binding protein is a selective RNA binding protein and the selective protein binding nucleic acid domain is a selective protein binding RNA domain. Optionally, the selective RNA binding protein and the selective protein binding nucleic acid domain are one or a combination of the following pairs: an MS2 coat protein (MCP) and an MS2 phage operator stem-loop, an RNA-binding section of the MCP and an MS2 phage operator stem-loop, a PP7 coat protein (PCP) and a PP7 phage operator stem-loop, an RNA-binding section of the PCP and a PP7 phage operator stem-loop, a Ku protein and a telomerase Ku binding motif, an RNA-binding section of the Ku protein and a telomerase Ku binding motif, an Sm7 protein and a telomerase Sm7 binding motif, an RNA-binding section of the Sm7 protein and a telomerase Sm7 binding motif, a Com RNA binding protein and a SfMu phage Com stem-loop, an RNA-binding section of the Com RNA binding protein and a SfMu phage Com stem-loop, and an aptamer ligand and a corresponding non-natural RNA aptamer, and an RNA-binding section of an aptamer ligand and a corresponding non-natural RNA aptamer. Optionally, (i) the selective RNA binding protein includes a MCP and the selective protein binding RNA domain includes a MS2 phage operator stem-loop, or (ii) the selective RNA binding protein includes a PP7 coat protein (PCP) and the selective protein binding RNA domain includes a PP7 phage operator stem-loop.

In another embodiment, the vesicle-, synapse- and/or organelle-associated protein or the polypeptide sequence that binds the vesicle-, synapse- and/or organelle-associated protein is a synaptic vesicle marker, a presynaptic synapse marker, a postsynaptic synapse marker, a ribosomal marker, a gap junction marker, a lysosomal marker, or an endosomal marker. Optionally, the vesicle-, synapse- and/or organelle-associated protein or the polypeptide sequence that binds the vesicle-, synapse- and/or organelle-associated protein is a protein having a synaptophysin domain, a protein having a fibronectin intrabody, an α-synuclein-binding FingR, a Bassoon-binding FingR a PSD95-binding FingR, or a GPHN-binding FingR.

In certain embodiments, the selective RNA binding protein and the selective protein binding nucleic acid domain include a zinc finger-based transcriptional regulation system. Optionally, the zinc finger-based transcriptional regulation system is capable of inhibiting further transgene expression once trafficking sites for the fusion protein are saturated. Optionally, the zinc finger-based transcriptional regulation system includes an MS2 binding protein.

In one embodiment, the other nucleic acid is an oligoribonucleotide. Optionally, the other nucleic acid is a therapeutic RNA and/or a transcript sequence.

In another embodiment, the oligoribonucleotide or oligonucleotide including a selective protein binding nucleic acid domain and a barcode nucleic acid or other nucleic acid is stabilized. Optionally, the oligoribonucleotide or oligonucleotide is stabilized by inclusion of one or more stabilizing modification or stabilizing sequence(s) including a polyA tail, a Murray Valley Encephalitis (MVE) pseudoknot, and/or nucleic acid (e.g., RNA) circularization. Optionally, the oligoribonucleotide or oligonucleotide is stabilized by inclusion of two or more MVE pseudoknots.

In some embodiments, the cell, tissue or organism is a mammalian cell, tissue or organism. Optionally, the mammalian cell is a neuron. Optionally, the mammalian cell is a cell in vivo.

In one embodiment, the composition further includes a viral vector. Optionally, the viral vector is a non-toxic viral vector. Optionally, the non-toxic viral vector is an Adeno-associated virus (AAV), an adenovirus or a lentivirus.

In another embodiment, the first plasmid and the second plasmid express non-toxic levels of the fusion protein and the oligoribonucleotide in the cell, tissue, or organism.

In certain embodiments, the barcode is at least 15 nucleotides in length. Optionally, the barcode also is degenerate and has at least a 2-3 base encoding barcodes at each barcode residue. Optionally, the barcode is of a length and level of degeneracy sufficient to produce a theoretical population of greater than 10⁹ unique barcodes. Optionally, the barcode is of a length and level of degeneracy sufficient to produce a theoretical population of greater than 10¹⁰ unique barcodes. Optionally, the barcode is of a length and level of degeneracy sufficient to produce a theoretical population of greater than 10¹¹ unique barcodes.

Another aspect of the instant disclosure provides a mammalian cell including a composition disclosed herein. In a related aspect, the instant disclosure provides a virus including a composition of the instant disclosure. Optionally, the virus is a non-toxic virus for infection of mammalian cells. Optionally, the non-toxic virus is an Adeno-associated virus (AAV), an adenovirus or a lentivirus.

Another aspect of the instant disclosure provides a method for detecting the localization of a fusion protein, vesicle and/or organelle in a cell, tissue or organism, the method involving: a) administering a composition or virus of the instant disclosure to the cell, tissue or organism; b) providing conditions suitable for fusion protein expression, binding of the oligonucleotide to the fusion protein, and time sufficient for localization of the bound oligonucleotide in the cell, tissue or organism to occur; and c) applying a spatially-localized sequencing assay or platform to at least a portion of the cell, tissue or organism, thereby obtaining sufficient sequence and location information to detect the localization of a barcode sequence within the cell, tissue or organism, thereby detecting the localization of the fusion protein, vesicle and/or organelle in the cell, tissue or organism.

In one embodiment, the spatially-localized sequencing assay or platform includes obtaining a tissue section (optionally a cryosection or a fixed tissue section) of the cell, tissue or organism and contacting the tissue section with a tagged array that retains sequence information while NGS sequencing is performed (the “SLIDE-seq” process). Optionally, the tagged array is a bead array capable of RNA capture and reconstruction of spatial localization of individual beads of the bead array.

In certain embodiments, the method further includes obtaining single-cell sequence/transcript profiling (e.g., single nucleus sequencing, snRNA-seq). Optionally, such single-cell sequence/transcript profiling is used as a comparator for a sequence obtained from the spatially-localized sequencing assay or platform. Optionally, the single-cell sequence/transcript profiling obtains a sequence of an injection site (e.g., for comparison to an in situ sequence, e.g., in situ barcodes at projection sites).

In another embodiment, the spatially-localized sequencing assay or platform includes contacting the cell, tissue or organism with a first monomer or linear polymer and a cross-linking agent including a second monomer or polymer, where the cross-linking agent is capable of crosslinking with the first monomer or linear polymer when combined (allowing for bridge amplification as a process for the generation of clusters of identical DNA, also referred to herein as “polymerization colonies”, or “PONIs”). Optionally, the method further involves contacting the cell, tissue or organism with a nucleic acid primer or probe harboring a modification capable of binding or chemically conjugating the primer or probe to the first monomer or linear polymer, the cross-linking agent, or both. Optionally, the first monomer or linear polymer includes one or more of the following compounds: acrylamide, methacrylate, polyethylene glycol (PEG), carboxymethyl cellulose (CMC), polyvinylpyrrolidone (PVP), isopropylacrylamide, hyaluronic acid, heparin, polylactic acid (PLA), polyglycolide (PGA), and poly(lactic-co-glycolic acid) (PLGA), Polyhydroxyalkanoates (PHA), propylene fumarate (PPF), agarose, alginate, chitosan, ethylene glycol-decorated polyisocyanide (PIC) polymers, derivatives thereof, and combinations thereof. Optionally, the cross-linking agent includes one or more of the following compounds: N,N′-methylene bisacrylamide, trisacrylamide, tetracrylamide, polyethylene glycol dimethacrylate, amine end-functionalized 4-arm star-PEG, derivatives thereof, and combinations thereof.

In one embodiment, application of the spatially-localized sequencing assay or platform includes obtaining single-cell sequence/transcript profiling (e.g., single nucleus sequencing, snRNA-seq).

In certain embodiments, the cell, tissue or organism or a tissue section of the cell, tissue or organism is contacted with a gapped padlock probe, where the gapped padlock probe targets the AAV barcode transcript to fill in the barcode sequence. Optionally, the method further includes ligating the gapped padlock probe including the barcode sequence and generating rolling circle colonies (“rolonies”) in situ.

In some embodiments, the cell, tissue or organism, or the tissue section of the cell, tissue or organism is fixed and/or permeabilized.

In a related embodiment, the tissue section of the cell, tissue or organism is a cryosection or a fixed tissue section. Optionally, the fixed tissue section is a formalin-fixed tissue section. Optionally, the formalin-fixed tissue section is a formalin-fixed paraffin-embedded (FFPE) tissue section. Optionally, the FFPE tissue section has been treated with xylene to remove paraffin.

In an additional embodiment, the method includes detecting both the barcode sequence and localization information for the barcode sequence in the cell, tissue or organism.

In one embodiment, the spatially-localized sequencing assay or platform is applied to a pre-synaptic neuron.

In another embodiment, the spatially-localized sequencing assay or platform is applied to a post-synaptic neuron. Optionally, the spatially-localized sequencing assay or platform is applied to an excitatory post-synaptic neuron.

In some embodiments, the spatially-localized sequencing assay or platform is applied to a cell that forms a chemical synapse or an electrical synapse.

In certain embodiments, the spatially-localized sequencing assay or platform is applied to a cell that forms a gap junction.

In another embodiment, the spatially-localized sequencing assay or platform is applied to a cell that forms a δ-Notch immune synapse.

In one embodiment, the method further includes performing single cell transcript profiling upon the cell, tissue or organism.

In certain embodiments, the spatially-localized sequencing assay or platform includes a quantitative spatial oligonucleotide sequencing system (detection system).

In a related embodiment, the barcode sequence is detected with spatial resolution of about 10 μm or less. Optionally, the barcode sequence is detected with spatial resolution of about 1 μm or less. Optionally, the barcode sequence is detected with spatial resolution of about 250 nm or less.

In another embodiment, the method further includes determining spatial proximity of two or more barcode sequences or other nucleic acids by measuring the frequency of recombination events between amplicons of the two or more barcode sequences or other nucleic acids during performance of bridge amplification. Optionally, spatial proximity of the two or more barcode sequences or other nucleic acids is detected at a neuronal synapse.

Another aspect of the instant disclosure provides a method for delivering a barcode nucleic acid or other nucleic acid to a subcellular compartment or organelle of a mammalian cell, the method including contacting the mammalian cell with a viral vector including: a) a first plasmid capable of being expressed in the cell, where the first plasmid encodes for the fusion protein, where the fusion protein includes a first domain including a subcellular compartment and/or organelle-associated protein or a polypeptide sequence that binds a subcellular compartment and/or organelle-associated protein and a second domain including a selective nucleic acid binding protein; and b) i) a second plasmid capable of being expressed in the cell, where the second plasmid encodes for an oligoribonucleotide including a selective protein binding nucleic acid domain, and a barcode nucleic acid or other nucleic acid, where the selective protein binding nucleic acid domain is capable of binding the selective nucleic acid binding protein encoded for by the first plasmid; or ii) an oligonucleotide including a selective protein binding nucleic acid domain, and a barcode nucleic acid or other nucleic acid, where the selective protein binding nucleic acid domain is capable of binding the selective nucleic acid binding protein encoded for by the first plasmid; under suitable conditions for intracellular trafficking and localization of the barcode nucleic acid to occur, thereby delivering the barcode nucleic acid or other nucleic acid to a subcellular compartment or organelle of the mammalian cell.

In a related embodiment, the subcellular compartment is a synaptic vesicle, a presynaptic synapse, a postsynaptic synapse, a ribosome, a gap junction, a lysosome, or an endosome. Optionally, the subcellular compartment is a synaptic vesicle.

A further aspect of the instant disclosure provides a kit including a composition, mammalian cell, or virus as disclosed herein and instructions for its use.

### Definitions

Unless specifically stated or obvious from context, as used herein, the term “about” is understood as within a range of normal tolerance in the art, for example within 2 standard deviations of the mean. “About” can be understood as within 10%, 9%, 8%, 7%, 6%, 5%, 4%, 3%, 2%, 1%, 0.5%, 0.1%, 0.05%, or 0.01% of the stated value.

In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Unless otherwise clear from context, all numerical values provided herein are modified by the term “about.”

As used herein, the term “amplification,” when used in reference to a nucleic acid, means copying the nucleic acid, wherein the copy has a nucleotide sequence that is the same as or complementary to at least a portion of the nucleotide sequence of the nucleic acid.

As used herein, the term “primer” when used in reference to a nucleic acid means a short nucleic acid sequence that provides a starting point for nucleic acid (e.g., DNA) synthesis. In some embodiments, primers are tagged with barcodes or unique molecular identifiers (UMIs).

As used herein, the term “amplicon,” when used in reference to a nucleic acid, means the product of copying the nucleic acid, wherein the product has a nucleotide sequence that is the same as or complementary to at least a portion of the nucleotide sequence of the nucleic acid. An amplicon can be produced by any of a variety of amplification methods that use the nucleic acid, or an amplicon thereof, as a template including, for example, bridge amplification, polymerase extension, polymerase chain reaction (PCR), rolling circle amplification (RCA), multiple displacement amplification (MDA), ligation extension, or ligation chain reaction. An amplicon can be a nucleic acid molecule having a single copy of a particular nucleotide sequence (e.g., a PCR product) or multiple copies of the nucleotide sequence (e.g., a recombination product of bridge amplification). A first amplicon of a target nucleic acid is typically a complementary copy. Subsequent amplicons are copies that are created, after generation of the first amplicon, from the target nucleic acid or from the first amplicon. A subsequent amplicon can have a sequence that is substantially complementary to the target nucleic acid or substantially identical to the target nucleic acid.

As used herein, the term “array” refers to a population of features or sites that can be differentiated from each other according to relative location. Different molecules that are at different sites of an array can be differentiated from each other according to the locations of the sites in the array. An individual site of an array can include one or more molecules of a particular type. For example, a site can include a single target nucleic acid molecule having a particular sequence or a site can include several nucleic acid molecules having the same sequence (and/or complementary sequence, thereof).

As used herein, the term “attached” refers to the state of two things being joined, fastened, adhered, connected or bound to each other. For example, an analyte, such as a nucleic acid, can be attached to a material, such as a gel or matrix, by a covalent or non-covalent bond. A covalent bond is characterized by the sharing of pairs of electrons between atoms. A non-covalent bond is a chemical bond that does not involve the sharing of pairs of electrons and can include, for example, hydrogen bonds, ionic bonds, van der Waals forces, hydrophilic interactions and hydrophobic interactions.

As used herein, the term “barcode sequence” is intended to mean a series of nucleotides in a nucleic acid that can be used to identify the nucleic acid, a characteristic of the nucleic acid (e.g., the identity and optionally the location of a bead to which the nucleic acid is attached), or a manipulation that has been carried out on the nucleic acid. In some embodiments the barcode is known as a unique molecular identifier (UMI). The barcode sequence can be a naturally occurring sequence or a sequence that does not occur naturally in the organism from which the barcoded nucleic acid was obtained. A barcode sequence can be unique to a single nucleic acid species in a population or a barcode sequence can be shared by several different nucleic acid species in a population. By way of further example, each nucleic acid probe in a population can include different barcode sequences from all other nucleic acid probes in the population. Alternatively, each nucleic acid probe in a population can include different barcode sequences from some or most other nucleic acid probes in a population. For example, each probe in a population can have a barcode that is present for several different probes in the population even though the probes with the common barcode differ from each other at other sequence regions along their length. In particular embodiments, one or more barcode sequences that are used with a biological specimen (e.g., a tissue sample) are not present in the genome, transcriptome or other nucleic acids of the biological specimen. For example, barcode sequences can have less than 80%, 70%, 60%, 50% or 40% sequence identity to the nucleic acid sequences in a particular biological specimen.

As used herein, the term “bridge amplification,” refers to an amplification method first exemplified in U.S. Ser. No. 12/774,126, which is incorporated herein by reference in its entirety. As employed herein, bridge amplification is a process for the generation of clusters of identical DNA, also referred to herein as “polymerization colonies”, or “PONIs”, to a target of interest.

As used herein, the term “cross-linking agent” refers to a molecule capable of bioconjugation to form a branched polymer matrix. “Cross-linking agents” are bifunctional agents containing reactive end groups that respond to functional groups, e.g., primary amines, carboxyls, sulfhydryls and carbonyls. The bifunctional agents are characterized as either homobifunctional or heterobifunctional, allowing for the formation of intermolecular and intramolecular crosslinking. In some embodiments, the cross-linking agent is selected from among the following: polyethylene glycol dimethacrylate, optionally triethylene glycol dimethyacrylate) (TEGDMA) or tetra(ethylene glycol) dimethacrylate, N,N′-methylene bisacrylamide, trisacrylamide, tetracrylamide, amine end-functionalized 4-arm star-PEG, derivatives thereof, and combinations thereof.

As used herein, the terms “monomer” or “linear polymer” when referring to a matrix composition means a precursor to an exogenously derived in situ matrix, optionally where the matrix is cross-linked to a preferred degree (optionally based upon the amount of input crosslinking agent and/or initiator compositions, crosslinking catalysts, or other components). In some embodiments, the monomer or linear polymer is selected from among the following: acrylamide, methacrylate, polyethylene glycol (PEG), carboxymethyl cellulose (CMC), polyvinylpyrrolidone (PVP), isopropylacrylamide, hyaluronic acid, heparin, PLA (polylactic acid), PGA (polyglycolide), and PLGA (poly(lactic-co-glycolic acid)), PHA (Polyhydroxyalkanoates), PPF (propylene fumarate), agarose, alginate, chitosan, ethylene glycol-decorated polyisocyanide (PIC) polymers, derivatives thereof, and combinations thereof.

As used herein, the term “in situ matrix” refers to a matrix polymerized in situ. In certain embodiments, the in situ matrix is suitable for providing a scaffold for enzymatic reactions. In some embodiments the in situ matrix is both porous and with sufficient structural integrity to covalently bind nucleic acids, e.g., primers or other molecules of interest, while retaining a level of spatial positioning sufficient to allow for spatial positioning of matrix-associated reactions to be obtained at some level of resolution (e.g., 100 μm or less, or other appropriate value of spatial resolution). In some embodiments, a matrix-associated enzymatic reaction is nucleic acid amplification. In some embodiments, the matrix can be polymerized via incubation at a temperature of 4° C. or 37° C., optionally at 4° C. and then 37° C., optionally repeating the temperature incubation steps 1, 2, 3, 4, or 5 times, optionally adding an initiator composition, optionally where the initiator composition is ammonium persulfate (APS) and tetramethylethylenediamine (TEMED), optionally wherein the initiator composition is riboflavin and TEMED.

As used herein, the term “spatial proximity information” refers to the relative spatial relationship of two molecules. In some embodiments, the two molecules are tagged with barcodes. In some exemplary embodiments, spatial proximity information is recorded through amplicons combining with neighboring sequences during bridge amplification. The closer the two sequences, the more likely they are to be recombined on the same amplicon. As described in Weinstein et al. (DNA Microscopy: Optics-free Spatio-genetic Imaging by a Stand-Alone Chemical Reaction. Cell. vol 178 (1) 2019), an algorithm decodes molecular proximities from the recombined sequences and infers physical images of the original transcripts at cellular resolution with precise sequence information. Spatial proximity information may be determined for PONIs using this method in any tissue, with an exemplary embodiment being detecting macromolecule spatial proximities in the vicinity of individual synapses in situ.

By “control” or “reference” is meant a standard of comparison. Methods to select and test control samples are within the ability of those in the art. Determination of statistical significance is within the ability of those skilled in the art, e.g., the number of standard deviations from the mean that constitute a positive result.

As used herein, the term “cryosection” refers to a piece of tissue, e.g., a biopsy, that has been obtained from a subject, snap frozen, embedded in optimal cutting temperature embedding material, frozen, and cut into thin sections. In certain embodiments, the thin sections can be fixed and permeabilized, optionally prior to adding a matrix-forming solution, e.g., in which a branched polymer with bound amplification primers polymerizes in situ.

As used herein, the term “different,” when used in reference to nucleic acids, means that the nucleic acids have nucleotide sequences that are not the same as each other. Two or more nucleic acids can have nucleotide sequences that are different along their entire length. Alternatively, two or more nucleic acids can have nucleotide sequences that are different along a substantial portion of their length. For example, two or more nucleic acids can have target nucleotide sequence portions that are different for the two or more molecules while also having a universal sequence portion that is the same on the two or more molecules.

As used herein, the term “each,” when used in reference to a collection of items, is intended to identify an individual item in the collection but does not necessarily refer to every item in the collection. Exceptions can occur if explicit disclosure or context clearly dictates otherwise.

As used herein, the term “extend,” or “polymerize” when used in reference to a nucleic acid, is intended to mean addition of at least one nucleotide or oligonucleotide to the nucleic acid. In particular embodiments one or more nucleotides can be added to the 3′ end of a nucleic acid, for example, via polymerase catalysis (e.g., DNA polymerase, RNA polymerase or reverse transcriptase). Chemical or enzymatic methods can be used to add one or more nucleotide to the 3′ or 5′ end of a nucleic acid. One or more oligonucleotides can be added to the 3′ or 5′ end of a nucleic acid, for example, via chemical or enzymatic (e.g., ligase catalysis) methods. A nucleic acid can be extended in a template directed manner, whereby the product of extension is complementary to a template nucleic acid that is hybridized to the nucleic acid that is extended.

As used herein, the term “next-generation sequencing” or “NGS” can refer to sequencing technologies that have the capacity to sequence polynucleotides at speeds that were unprecedented using conventional sequencing methods (e.g., standard Sanger or Maxam-Gilbert sequencing methods). In some embodiments, NGS is performed after in situ bridge amplification PONIs are released from the tissue. The unprecedented speeds of NGS are achieved by performing and reading out thousands to millions of sequencing reactions in parallel. NGS sequencing platforms include, but are not limited to, the following: Massively Parallel Signature Sequencing (Lynx Therapeutics); 454 pyro-sequencing (454 Life Sciences/Roche Diagnostics); solid-phase, reversible dye-terminator sequencing (Solexa/Illumina™); SOLID™ technology (Applied Biosystems); Ion semiconductor sequencing (Ion Torrent™); and DNA nanoball sequencing (Complete Genomics). Descriptions of certain NGS platforms can be found in the following: Shendure, et al., “Next-generation DNA sequencing,” Nature, 2008, vol. 26, No. 10, 1135-1145; Mardis, “The impact of next-generation sequencing technology on genetics,” Trends in Genetics, 2007, vol. 24, No. 3, pp. 133-141; Su, et al., “Next-generation sequencing and its applications in molecular diagnostics” Expert Rev Mol Diagn, 2011, 11 (3): 333-43; and Zhang et al., “The impact of next-generation sequencing on genomics”, J Genet Genomics, 201, 38 (3): 95-10⁹.

As used herein, the terms “nucleic acid” and “nucleotide” are intended to be consistent with their use in the art and to include naturally occurring species or functional analogs thereof. Particularly useful functional analogs of nucleic acids are capable of hybridizing to a nucleic acid in a sequence specific fashion or capable of being used as a template for replication of a particular nucleotide sequence.

Naturally occurring nucleic acids generally have a backbone containing phosphodiester bonds. An analog structure can have an alternate backbone linkage including any of a variety of those known in the art. Naturally occurring nucleic acids generally have a deoxyribose sugar (e.g., found in deoxyribonucleic acid (DNA)) or a ribose sugar (e.g., found in ribonucleic acid (RNA)). A nucleic acid can contain nucleotides having any of a variety of analogs of these sugar moieties that are known in the art. A nucleic acid can include native or non-native nucleotides. In this regard, a native deoxyribonucleic acid can have one or more bases selected from the group consisting of adenine, thymine, cytosine or guanine and a ribonucleic acid can have one or more bases selected from the group consisting of uracil, adenine, cytosine or guanine. Useful non-native bases that can be included in a nucleic acid or nucleotide are known in the art. The terms “probe” or “target,” when used in reference to a nucleic acid or sequence of a nucleic acid, are intended as semantic identifiers for the nucleic acid or sequence in the context of a method or composition set forth herein and does not necessarily limit the structure or function of the nucleic acid or sequence beyond what is otherwise explicitly indicated. The terms “probe” and “target” can be similarly applied to other analytes such as proteins, small molecules, cells or the like.

As used herein, the term “subject” includes humans and mammals (e.g., mice, rats, pigs, cats, dogs, and horses). In many embodiments, subjects are mammals, particularly primates, especially humans. In some embodiments, subjects are livestock such as cattle, sheep, goats, cows, swine, and the like; poultry such as chickens, ducks, geese, turkeys, and the like; and domesticated animals particularly pets such as dogs and cats. In some embodiments (e.g., particularly in research contexts) subject mammals will be, for example, rodents (e.g., mice, rats, hamsters), rabbits, primates, or swine such as inbred pigs and the like.

As used herein, the term “tissue” is intended to mean an aggregation of cells, and, optionally, intercellular matter. Typically, the cells in a tissue are not free floating in solution and instead are attached to each other to form a multicellular structure. Exemplary tissue types include nerve (e.g., brain/CNS), muscle, epidermal and connective tissues.

Unless specifically stated or obvious from context, as used herein, the term “or” is understood to be inclusive. Unless specifically stated or obvious from context, as used herein, the terms “a”, “an”, and “the” are understood to be singular or plural.

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it is understood that the particular value forms another aspect. It is further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. It is also understood that throughout the application, data are provided in a number of different formats and that this data represent endpoints and starting points and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 as well as all intervening decimal values between the aforementioned integers such as, for example, 1.1, 1.2, 1.3, 1.4, 1.5, 1.6, 1.7, 1.8, and 1.9. With respect to sub-ranges, “nested sub-ranges” that extend from either end point of the range are specifically contemplated. For example, a nested sub-range of an exemplary range of 1 to 50 may comprise 1 to 10, 1 to 20, 1 to 30, and 1 to 40 in one direction, or 50 to 40, 50 to 30, 50 to 20, and 50 to 10 in the other direction.

The transitional term “comprising,” which is synonymous with “including,” “containing,” or “characterized by,” is inclusive or open-ended and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention.

The embodiments set forth below and recited in the claims can be understood in view of the above definitions.

Other features and advantages of the disclosure will be apparent from the following description of the preferred embodiments thereof, and from the claims. Unless otherwise defined, all technical and scientific terms used herein have the same meaning as commonly understood by one of ordinary skill in the art to which this disclosure belongs. Although methods and materials similar or equivalent to those described herein can be used in the practice or testing of the present disclosure, suitable methods and materials are described below. All published foreign patents and patent applications cited herein are incorporated herein by reference. All other published references, documents, manuscripts and scientific literature cited herein are incorporated herein by reference. In the case of conflict, the present specification, including definitions, will control. In addition, the materials, methods, and examples are illustrative only and not intended to be limiting.

### BRIEF DESCRIPTION OF THE DRAWINGS

The following detailed description, given by way of example, but not intended to limit the disclosure solely to the specific embodiments described, may best be understood in conjunction with the accompanying drawings, in which:

FIG. 1A shows a schematic that outlines the general strategy of tagging presynaptic boutons with exogenous mRNA. A synaptic trafficking protein and an mRNA reporter are jointly introduced to the same neuron. The trafficking protein is a fusion of a synaptophysin domain, which shuttles to presynaptic boutons, and the MS2 protein, which binds to a specific RNA stem loop motif. The mRNA reporter transcript contains tandem MS2 stem loop repeats in the 3′ UTR. When introduced into the same neuron, the stem-loop-containing mRNA reporter transcript binds tightly to MS2, enabling the RNA to be shuttled to the presynaptic compartment through the natural trafficking of the synaptophysin domain.

FIG. 1B shows images of in vitro evidence for presynaptic transport of mRNA, each bouton labelled with a white arrow. Simultaneous single-molecule fluorescence in situ hybridization (smFISH) for the mRNA reporter transcript, and imaging of the fluorescent presynaptic targeting protein demonstrated highly sensitive and specific trafficking of transcripts to presynaptic compartments.

FIG. 1C shows a drawing and immunofluorescent images that demonstrate the in vivo validation of presynaptic mRNA transport by label in CA3-to-RSP projections.

FIG. 1D depicts a bar graph that shows quantification of in vivo synaptic labeling efficiency of the experiment shown in FIG. 1C.

FIG. 2A depicts an image that shows with arrows that synaptophysin is present in the axon outside of terminals.

FIG. 2B depicts an image that shows that alpha-Synuclein FingR does not show accumulation outside of terminals.

FIG. 2C depicts an image that demonstrates that synaptophysin staining and alpha-synuclein FingR accumulate at synapses, shown by arrowheads.

FIG. 3A shows the structure of the 33-base pair barcode leading to enormous diversity (5′-WSBWSDWSHWSVWSBWSWSHWSVWSBWSDWSH-3′, SEQ ID NO: 12). The IUPAC code is shown for reference.

FIG. 3B depicts a schematic that shows the location of the barcode with the synaptically localized mRNA.

FIG. 3C depicts an image/illustration that shows the location of VPM, the thalamic region that receives somatosensory input.

FIG. 3D depicts a graph that shows a UMAP representation of neurons extracted from an scRNAseq experiment performed on VPM injected with presynaptic trafficking system and barcoded mRNA. Clear diversity of cell states and types is observed.

FIG. 3E shows three graphs that demonstrate the validation of neuronal cell-type diversity in the injected VPM. From left to right, the graphs show the normalized gene count for Gadl, Slc17a6, and Prkcd.

FIG. 3F shows three images of in-situ labelling of the same three genes that highlights their expression in the thalamus (data from Allen Brain Atlas).

FIG. 4A depicts a UMAP representation that demonstrates the subclustering of excitatory neurons from the full dataset. Broad scaled expression of the mRNA reporter is observed.

FIG. 4B depicts a graph that shows the number of unique barcodes for all excitatory neurons.

FIG. 4C depicts a histogram that shows the frequency of a barcode over one or more cells for all excitatory neurons.

FIG. 5A depicts an image that shows the expression of exogenous viral mRNA, that was detected on a Slide-seq assay performed on a mouse injected with barcoded AAV in the LGN.

FIG. 5B shows four images that demonstrate the expression of markers of different cortical cell types. Clockwise from top left, they are cortical layer 6B (Ctgf), cortical layer 2/3 (Cux2), Oligodendrocytes (Mbp) and cortical layer 4 (Rorb).

FIG. 5C depicts a graph that shows the results of the targeted amplification protocol to enrich and sequence in parallel the transported mRNA.

FIG. 6A shows a schematic of postsynaptic editing using a nanobody.

FIG. 6B depicts an immunofluorescent image that shows a primary cortical neuron transfected with the targeting protein (Nanobody-GFP-MS2 RNA stem loop binding domain) and the mRNA barcode (RNA pseudoknot-mScarlet CDS-MS2 RNA stem loops).

FIG. 6C shows a graph that demonstrates the percentage of trafficking efficiency measured by total postsynaptic spines labelled by mRNA.

FIG. 6D shows an immunofluorescent image that demonstrates that postsynaptic targeting protein colocalizes with Homer in the hippocampus in vivo.

FIG. 6E depicts an image/illustration and accompanying images showing that AAV delivery of postsynaptic system in-vivo hippocampus reveals robust trafficking into synaptic layers of hippocampus (syn) which is not observed in absence of stem loops.

FIG. 6F shows an image of a primary cortical neuron expressing the inhibitory postsynaptic system which labels Gephyrin-positive spines.

FIG. 7 shows two images that demonstrate synaptosome isolations showing colocalization of synaptic targeting proteins. The top image is a broad field of view and a single synaptosome view of a synaptosome contained postsynaptic targeting protein (GFP), co-stained with a pre- and post-synaptic marker. Synapsin is the pre-synaptic marker, and PSD95 is the post-synaptic marker. The bottom image represents the same field of view as the top image, but from tissue transduced with presynaptic targeting protein.

FIG. 8 shows an image of fluorescent probes bound to in situ rolonies where 12 bases were gap-filled in situ in fixed Hela cells. Each puncta represents a single rolony. The nuclei are shown in light grey.

FIG. 9 shows an image of one base of in situ sequencing of rolonies that are generated in the nucleus. Each variation of shading-dark grey, medium grey, very light grey, light grey-represents a different base identity. It is observed that, despite that the rolonies are volume filling, few individual rolonies are mixtures of colors.

FIG. 10 depicts a schematic showing, at top left, the incorporation of primers into a polymer network. At bottom left, the bridge amplification of targeted molecules is shown, and the schematic to the right schematic shows the mechanism of interaction quantification through heteroduplex formation and overlap extension.

FIG. 11 depicts a series of images that show smFISH detection of PONI amplicons. Amplicons of the gene Hpca within the hippocampus are shown with amplification primers in the top four images, and without amplification primers in the bottom three images. All images are shown across different numbers of bridge amplification cycles.

FIG. 12 shows images of untargeted PONI amplification. At top right, smFISH detection of PONI is shown. At the bottom right, the same tissue section is shown, stained for PONI amplicons of Mbp. Images from Allen Brain Atlas are on the left, that show the expected expression patterns in the box inset.

FIG. 13A shows two graphs that show plots of recombination counts between cDNA and antibodies to either RBFOX3 in the top graph, or OLIG2 in the bottom graph.

FIG. 13B shows a graph that represents the percentage of intronic alignments of recombinant reads between cDNA and four different antibodies. H3 and RBFOX3 are nuclear antibodies, and GFAP and SYN are cytoplasmic antibodies.

FIG. 14 shows a schematic of a plasmid DNA that contains (encodes for) a protein component packaged in one virion and a plasmid containing (encoding for) an RNA component packaged in another virion. On the left, the protein component fuses a trafficking protein and a selective RNA binding protein. On the right, the RNA component fuses a selective protein bonding RNA and an RNA barcode.

FIG. 15 depicts an illustration showing packaged viruses introduced to a live animal such that the protein component and the RNA component will start expressing inside cells.

FIG. 16 depicts a schematic showing how the selective protein binding RNA binds to the selective RNA binding protein, bringing the protein component and the RNA component together.

FIG. 17 depicts an illustration that demonstrates that the combined components are subsequently trafficked to specific subcellular compartments via the trafficking protein.

FIG. 18 shows an illustration of neuronal cell regions, including an extended axon. The region in which the virus is delivered is called the injection site. The region in which the combined components are trafficked to is called the projection site. If the injection site and the projection site are close by, the single-cell transcriptome and the trafficked RNA Barcodes can both be sequenced via in situ sequencing. If the injection site and the projection site are far apart, the single-cell transcriptome and the trafficked RNA Barcodes can also be sequenced via in situ sequencing. Alternatively, if the injection site and the projection site are far apart, the injection site can be dissected and sequenced using single cell dissociation-based single-cell sequencing and the Projection Site can be sequenced separately using in situ sequencing or spatial transcriptomes.

FIG. 19 shows a schematic of the intracranial injection of the Synapse-seq trafficking system. In the left image/drawing, virions (V1) are injected into the dorsolateral geniculate nucleus (dLGN) region of the brain of C57BL/6 mice, located in the thalamus. At top left, a graph shows a UMAP representation of the results of the viral injection, utilizing snRNA-seq to read out mRNA barcodes in neuronal nuclei. Images at the middle left show successful trafficking of mRNA barcodes to dLGN, validated by in situ hybridization. The images show from left to right, successful trafficking of the targeting protein, the mRNA barcode, and an image of their overlap. The bottom right image shows that Slide-seq was used to read out synaptically trafficked mRNA barcodes, with images shown in projection from the dLGN. The barcodes read out were Slc17a7, Tcf 12, L6 CT, L4/5/6 IT, and L2/3 IT.

FIGS. 20A-20C depict the application of Synapse-seq to long-range corticothalamic projections. FIG. 20A shows the primary visual cortex (VISP) targeted for snRNAseq, and the dorsal lateral geniculate nucleus of the thalamus (dLGN) targeted for Slide-seq. FIG. 20B depicts an image that shows the VISP projection neuron snRNAseq data. FIG. 20C shows an example of a Slide-seq section from dLGN. Tef7 2 is in the thalamus.

FIG. 21 depicts an image that shows matched barcodes from each of the pyramidal neuron clusters within the LGN region of an example Slide-seq section, demonstrating the ability to map long-range projections in the brain.

### DETAILED DESCRIPTION OF THE INVENTION

The present disclosure is directed, at least in part, to the discovery of a molecular system that is capable of measuring connectivity of cells, in a manner that maintains the health of such cells throughout the process of introducing exogenous plasmids and/or other nucleic acids to the cells and then allowing the cells to express the exogenous plasmids and/or other nucleic acids, which are designed to intracellularly tag and traffic with subcellular compartments, organelles or other subcellular locations (e.g., neuronal synapses), until detection steps are performed. Certain aspects of the present disclosure provide a system termed “Synapse-seq” herein. In some embodiments, the Synapse-seq technology provides a set of diversely barcoded AAV-delivered transcripts that has recently been developed to be specifically and abundantly trafficked to either the presynaptic or postsynaptic compartments of neurons. As exemplified, the current disclosure employs this method of specific trafficking without major alterations in neuronal health or function, using a combination of AAV barcoded transcriptional reporters and existing high-throughput single cell and spatial readouts. Spatial transcriptomic technology has specifically been applied herein to deliver brain-wide projection neuron mapping, digital counts of postsynaptic spine densities on transcriptionally defined neurons, and to generate cell-type-specific connectivity networks. Each of these measurements represent a substantial improvement in the scale, feasibility, and quality over existing cell-type-specific connectomics measurements.

To provide both research and commercial value, tools for measuring cellular/neuronal connectivity should have the following properties: 1) they should be high throughput, enabling statistically robust synaptic measurements across many cell types; 2) they should simultaneously report synaptic information and cell type information, such as gene expression or epigenetic regulation, to link synaptic connectivity to molecular identity (such tools, while reporting this information, should also minimally disrupt endogenous gene regulation); 3) they should provide digital (quantitative) readouts of connectivity via synapse counts per cell, with high sensitivity and specificity; 4) they should provide as much spatial information as possible about axonal projections and the locations of synaptic connections, since the localization of synapses along processes influences their functions; and 5) they should be sufficiently flexible and cost-effective to be able to routinely phenotype many brains, across diverse genetic, behavioral, and pharmacological perturbations. The system disclosed herein, termed “Synapse-seq”, optimally incorporates these five key properties.

Synapse-seq contains many options that work together to promote these properties. It promotes high-throughput use, because the virus used for delivery can be any vector, including AAV and Php.eb. AAV-PHP.eB is a vector system that, along with enhanced CNS tropism, has been validated in gene delivery in vivo across the blood brain barrier following intravenous infusion. The RNA barcode in Synapse-seq, at least as currently exemplified, has been protected from degradation by incorporating a polyA tail and Murray Valley Encephalitis (MVE) pseudoknot, but can be protected using other means such as two MVE pseudoknots or RNA circularization. The plasmid DNA(s) of the instant disclosure can be delivered locally, systemically, or incorporated into a transgenic animal, allowing for flexibility. The trafficking (subcellular compartment and/or organelle-tagging) component can be changed to target any subcellular compartment, including synapses and axons, allowing for many applications across diverse conditions. The specific protein binding RNA and specific RNA binding protein can be changed into any pair with high specificity, including MS2 and PP7 stem-loop RNA labeling. MS2 and PP7 are bacteriophages with coat proteins. The binding of sequence-specific RNA-interacting proteins, such as the bacteriophage MS2 or PP7 coat proteins has been extremely useful and widely used to visualize single mRNAs in vivo.

The present disclosure provides innovation on at least the following three key fronts: First, a highly sensitive and specific mRNA trafficking system for delivering barcoded nucleic acids to each side of the synapse has been developed and is disclosed herein. In particular, the instant disclosure provides transcripts that are so efficiently brought to synaptic compartments, that they can be easily delivered using the most commonly used viral transduction system, AAV. AAV has minimal effects on neuronal cell health, and is easily produced and distributed, enabling the widespread use of this viral transduction system in neuroscience. Second, a purely AAV-based system, such as that provided by certain aspects and embodiments of the instant disclosure, can be used in any organism that can be transduced—i.e., the recent exciting development of CNS-wide transduction in non-human primates provides a direct opportunity to apply the Synapse-seq tools disclosed herein to macaque and marmoset circuits. Third, the instant disclosure describes adaptation and development of new high-throughput and in situ readouts for quantifying the accumulation of, e.g., synaptically localized barcoded transcripts, to map axonal projections, quantify synapses on cells, and measure cell-specific connectivity. These readouts represent significant improvements in the throughput of measuring such connections over existing methods. Certain previously described spatially-informed detection and readout technologies, particularly “Slide-seq” (see, e.g., PCT/US19/30194) and “PONI-seq” (see, e.g., PCT/US22/16144), the latter of which is capable of detecting molecular proximity in situ, can be used with the compositions and methods of the instant disclosure, to allow the compositions and methods of the instant disclosure not only to perform cell-cell connectivity measurements, but also additional biological assays, including in situ chromatin immunoprecipitation with antigen-specific antibodies combined with massively parallel sequencing (CHIP-seq), mapping gap junction connections between cells, and quantifying transcripts being actively translated on ribosomes in situ.

Next-generation DNA sequencing provides a digital, high-throughput measurement modality that was recognized herein as uniquely suited to mapping synaptic connections amongst cellular partners. In recent years, DNA sequencing has been leveraged to massively increase the throughput of single-cell RNA-seq, and these technologies have been applied to comprehensively survey cell type specialization in the mammalian brain. Moreover, DNA barcoding and sequencing technologies have been applied herein to the spatial analysis of gene expression to develop high-resolution (10 micron) maps of cell type locations within specific neuroanatomical nuclei, using the current Slide-seq methods. The current results have revealed enormous heterogeneity, on the order of thousands of individual neuronal types, many of which likely have distinct patterns of synaptic connectivity. At present, however, knowledge of the synaptic connections amongst these cell types remains largely unknown.

Large-scale connectomics projects to define brain circuitry in the nematode and fly have served as foundational hypothesis-generation tools for understanding structure-function relationships in those invertebrate model systems. A tool such as Synapse-seq of the instant disclosure, that can deliver similar measurements of neuronal connectivity in specific cell types within mammalian brains is viewed as enormously enabling to neuroscience, with applications both to basic understanding of neuronal function, and to understanding neuropsychiatric and neurodegenerative disease pathogenesis.

The potential for DNA sequencing to be used as a readout of synaptic connectivity has been appreciated for many years. Pioneering work by the Zador lab has used a high-expression Sindbis viral system to deliver barcoded transcripts to neurons that could be trafficked to axons, enabling neuronal projections to be mapped by bulk RNA sequencing of microdissected synaptic targets or in situ sequencing by gap-fill ligation. While useful for mapping some long-range projection neurons, this strategy does not localize transcripts specifically to presynaptic compartments (just to axons). More importantly, Sindbis exerts significant effects on the transcriptional state of the infected tissue. Combining monosynaptic-traced neurons after rabies infection with single-cell transcriptomics has linked connected neurons and more recently, been combined with high-diversity barcoding. Rabies suffers from toxicity problems similar to the Sindbis system, and a particular challenge of identifying starter cells. In addition, the spread of rabies has a complex and sometimes ambiguous relationship to true synaptic connectivity in vivo. Thus, the current Synapse-seq system provides significant advantages over these previously described attempts at identifying synaptic connectivity in mammalian cells.

The presynaptic trafficking system disclosed herein has been demonstrated to be highly effective in vivo. Presynaptic transcripts can be reliably detected and sequenced by Slide-seq. While the current strategy relies on the intersectional detection of barcodes in snRNA-seq (at the AAV injection source) and Slide-seq (at the projection target), it is contemplated that if the sampling of cells at the source and projections at the target are each sparse, the number of shared barcodes could be extremely sparse (since the probability is multiplicative). Solutions to such a situation include additionally scaling Slide-seq to hundreds of serial sections, if need be, to more densely and widely sample projections, which is actually not very expensive (since targeted sequencing of just the AAV transcript can be performed in the current process, significantly reducing current DNA sequencing needs). This process has been internally scaled dramatically since its initial description, but new industry products and innovations also make these experiments significantly more tractable. Another solution is to microdissect the somatosensory cortex, and perform bulk sequencing of barcodes from the dissectate. Although valuable spatial information will be lost in such a process, this would also enable the reconstruction of many more projections, based on results from other systems.

Additional details of the instant disclosure are provided in the following sections.

### Trafficking Proteins and Other Markers

Certain aspects of the instant disclosure employ trafficking proteins or other polypeptides capable of associating with or otherwise marking subcellular compartments (e.g., synaptic vesicles), organelles, or other locations within the cell. While any art-recognized trafficking protein or other intracellular marker may be employed in the currently disclosed Synapse-seq system, specific examples of such trafficking proteins or other markers include, without limitation, synaptophysin and synaptophysin-binding polypeptides, Bassoon (a presynaptic scaffolding protein) and Bassoon-binding polypeptides, PSD95 (Postsynaptic Density Protein 95, also known as DLG4 or Discs Large MAGUK Scaffold Protein 4-a postsynaptic protein found at excitatory synapses) and PSD95-binding polypeptides (including, e.g., PSD95-FingR), GPHN (Gephyrin-a postsynaptic protein found only at inhibitory synapses) and GPHN-binding polypeptides (including, e.g., GPHN-FingR), as well as, more generally, any synaptic vesicle marker, presynaptic synapse marker, postsynaptic synapse marker, ribosomal marker, gap junction marker, lysosomal marker, endosomal marker, etc. Proteins that include a synaptophysin domain, proteins that include a fibronectin intrabody, α-synuclein-binding FingR polypeptides, and other such proteins, are also specifically contemplated.

### RNA Binding Proteins

In certain aspects, the systems of the instant disclosure employ RNA binding proteins, as well as correspondingly bound RNA motifs. In such aspects, a nascently expressed fusion protein having a trafficking polypeptide (or other subcellular compartment/location-associated or—binding marker polypeptide) is fused with a RNA binding polypeptide, and presence of the RNA binding polypeptide allows for a highly avid/high affinity interaction between such a RNA binding polypeptide and a specific RNA motif. Pairs of RNA recognition motif (protein binding RNA sequences)/RNA binding protein can be derived from naturally occurring sources (e.g., RNA phages, or yeast telomerase) or can be artificially designed (e.g., RNA aptamers and their corresponding binding protein ligands). A non-exhaustive list of examples of Protein binding RNA domain/RNA binding protein pairs expressly contemplated for use in the currently disclosed Synapse-seq system is provided in Table 1, as well as in the following sequences.

TABLE 1
Exemplary selective RNA binding proteins and corresponding
selective protein binding RNA domains

| RNA binding protein | Protein binding RNA domain | Organism |
|---|---|---|
| MS2 Coat Protein (MCP) | MS2 phage operator stem-loop | Phage |
| PP7 coat protein (PCP) | PP7 phage operator stem-loop | Phage |
| Com RNA binding | SfMu phage Com stem-loop | Phage |
| protein | | |
| Ku | Telomerase Ku binding motif | Yeast |
| Sm7 | Telomerase Sm7 binding motif | Yeast |
| Corresponding | Non-natural RNA aptamer | Artificially |
| aptamer ligand | | designed |

1. MS2 phage operator stem loop/MS2 coat protein
a. MS2 phage operator

(SEQ ID NO: 1)
5′-GCGCACATGAGGATCACCCATGTGC-3′

b. MS2 coat protein

(SEQ ID NO: 2)
MASNFTQFVLVDNGGTGDVTVAPSNFANGIAEWISSNSRSQAYKV
TCSVRQSSAQNRKYTIKVEVPKGAWRSYLNMELTIPIFATNSDCE
LIVKAMQGLLKDGNPIPSAIAANSGIY

2. PP7 phage operator stem loop/PP7 coat protein
a. PP7 phage operator stem loop

(SEQ ID NO: 3)
5′-aTAAGGAGTTTATATGGAAACCCTTA-3′

b. PP7 coat protein (PCP)

(SEQ ID NO: 4)
MSKTIVLSVGEATRTLTEIQSTADRQIFEEKVGPLVGRLRLTASL
RQNGAKTAYRVNLKLDQADVVDCSTSVCGELPKVRYTQVWSHDVT
IVANSTEASRKSLYDLTKSLVATSQVEDLVVNLVPLGR

3. SfMu Com stem loop/SfMu Com binding protein
a. SfMu Com stem loop

(SEQ ID NO: 5)
5′-CTGAATGCCTGCGAGCATC-3′

b. SfMu Com binding protein

(SEQ ID NO: 6)
MKSIRCKNCNKLLFKADSFDHIEIRCPRCKRHIIMLNACEHPTEK
HCGKREKITHSDETVRY

4. Telomerase Ku biding motif Ku heterodimer
a. Ku binding hairpin

(SEQ ID NO: 7)
5′-TTCTTGTCGTACTTATAGATCGCTACGTTATTTCAATTTTGA
AAATCTGAGTCCTGGGAGTGCGGA-3′

b. Ku heterodimer

(SEQ ID NO: 8)
MSGWESYYKTEGDEEAEEEQEENLEASGDYKYSGRDSLIFLVDAS
KAMFESQSEDELTPFDMSIQCIQSVYISKIISSDRDLLAVVFYGT
EKDKNSVNFKNIYVLQELDNPGAKRILELDQFKGQQGQKRFQDMM
GHGSDYSLSEVLWVCANLFSDVQFKMSHKRIMLFTNEDNPHGNDS
AKASRARTKAGDLRDTGIFLDLMHLKKPGGFDISLFYRDIISIAE
DEDLRVHFEESSKLEDLLRKVRAKETRKRALSRLKLKLNKDIVIS
VGIYNLVQKALKPPPIKLYRETNEPVKTKTRTFNTSTGGLLLPSD
TKRSQIYGSRQIILEKEETEELKRFDDPGLMLMGFKPLVLLKKHH
YLRPSLFVYPEESLVIGSSTLFSALLIKCLEKEVAALCRYTPRRN
IPPYFVALVPQEEELDDQKIQVTPPGFQLVFLPFADDKRKMPFTE
KIMATPEQVGKMKAIVEKLRFTYRSDSFENPVLQQHFRNLEALAL
DLMEPEQAVDLTLPKVEAMNKRLGSLVDEFKELVYPPDYNPEGKV
TKRKHDNEGSGSKRPKVEYSEEELKTHISKGTLGKFTVPMLKEAC
RAYGLKSGLKKQELLEALTKHFQD>

(SEQ ID NO: 9)
MVRSGNKAAVVLCMDVGFTMSNSIPGIESPFEQAKKVITMFVQRQ
VFAENKDEIALVLFGTDGTDNPLSGGDQYQNITVHRHLMLPDFDL
LEDIESKIQPGSQQADFLDALIVSMDVIQHETIGKKFEKRHIEIF
TDLSSRFSKSQLDIIIHSLKKCDISERHSIEWPCRLTIGSNLSIR
IAAYKSILQERVKKTWTVVDAKTLKKEDIQKETVYCLNDDDETEV
LKEDIIQGFRYGSDIVPFSKVDEEQMKYKSEGKCFSVLGFCKSSQ
VQRRFFMGNQVLKVFAARDDEAAAVALSSLIHALDDLDMVAIVRY
AYDKRANPQVGVAFPHIKHNYECLVYVQLPFMEDLRQYMFSSLKN
SKKYAPTEAQLNAVDALIDSMSLAKKDEKTDTLEDLFPTTKIPNP
RFQRLFQCLLHRALHPREPLPPIQQHIWNMLNPPAEVTTKSQIPL
SKIKTLFPLIEAKKKDQVTAQEIFQDNHEDGPTAK

5. Telomerase Sm7 biding motif/Sm7 homoheptamer
a. Sm consensus site (single stranded)

(SEQ ID NO: 10)
5′-AATTTTTGGA-3′

b. Monomeric Sm-like protein (archaea)

(SEQ ID NO: 11)
GSVIDVSSQRVNVQRPLDALGNSLNSPVIIKLKGDREFRGVLKSF
DLHMNLVLNDAEELEDGEVTRRLGTVLIRGDNIVYISP

### Massively Diverse Barcode Nucleic Acid Libraries

As currently exemplified, the quantitative molecular trafficking/organelle detection system of the instant disclosure employs a 33 nucleotide nucleic acid barcode, whose theoretical diversity is on the order of 10¹¹ sequences (by synthesizing such sequences while allowing for 2-3 nucleotide options at any given nucleotide, across all 33 positions). This sequence diversity should be sufficient to uniquely barcode each AAV virion that is stereotactically injected into an animal, allowing for the tracking of single-cell-infected viruses in vivo. It is expressly contemplated that other barcode nucleic acid libraries could readily be used in the current system, including those with significantly less diversity (e.g., approx. 10⁹, 10⁸, 10⁷, 10⁶, 10⁵ or fewer sequences possible) or with significantly more diversity (e.g., approx. 10¹², 10¹³, 10¹⁴, 10¹⁵, 10¹⁶ or more sequences possible). Design and synthesis of such diverse barcode nucleic acid libraries is well known in the art.

### Transcriptional Regulation Systems

Certain embodiments of the instant disclosure employ transcriptional regulation systems, e.g., to minimize intracellular disruption that might otherwise be caused by expression of the Synapse-seq components in a target cell. For example, in certain embodiments, a presynaptic targeting protein of the current disclosure is fused to a zinc finger-based transcriptional regulation system, which has been described as capable of inhibiting further transgene expression once trafficking sites are saturated. Implementation of similar transcriptional control loops are envisioned also to generate a highly specific postsynaptic targeting system. In other embodiments, co-expression of a presynaptic targeting protein with a zinc finger self-repressor using a P2A self-cleaving peptide is contemplated. As will be apparent to the skilled artisan, any amenable transcriptional control loop system can also be employed with the current Synapse-seq system.

### Viral Vectors

Certain aspects of the instant disclosure employ viral vectors for nucleic acid delivery to living cells. Such viral vectors for gene delivery are widely known in the art and can include, e.g., Adeno-associated virus (AAV), adenovirus, and/or lentivirus, among other viral vectors known in the art. While the virus used for delivery can be any vector, certain embodiments, employ AAV, optionally AAV and Php.eb. AAV-PHP.eB is a vector system that, along with enhanced CNS tropism, has been validated in gene delivery in vivo across the blood brain barrier following intravenous infusion.

### Tissue Samples and Sectioning

In some embodiments, a tissue section is employed. The tissue can be derived from a multicellular organism. Exemplary multicellular organisms include, but are not limited to a mammal, plant, algae, nematode, insect, fish, reptile, amphibian, fungi or Plasmodium falciparum. Exemplary species are set forth previously herein or known in the art. The tissue can be freshly excised from an organism, or it may have been previously preserved for example by freezing, embedding in a material such as paraffin (e.g., formalin fixed paraffin embedded samples), formalin fixation, infiltration, dehydration or the like. Optionally, a tissue section can be cryosectioned, using techniques and compositions as described herein and as known in the art. As a further option, a tissue can be permeabilized and the cells of the tissue lysed. Any of a variety of art-recognized lysis treatments can be used. Target nucleic acids that are released from a tissue that is permeabilized can be captured by nucleic acid probes, as described herein and as known in the art.

A tissue can be prepared in any convenient or desired way for its use in a method, composition or apparatus herein. Fresh, frozen, fixed or unfixed tissues can be used. A tissue can be fixed or embedded using methods described herein or known in the art.

A tissue sample for use herein, can be fixed by deep freezing at temperature suitable to maintain or preserve the integrity of the tissue structure, e.g., less than −20° C. A fixed or embedded tissue sample can be sectioned, i.e., thinly sliced, using known methods. For example, a tissue sample can be sectioned using a chilled microtome or cryostat, set at a temperature suitable to maintain both the structural integrity of the tissue sample and the chemical properties of the nucleic acids in the sample. Exemplary additional fixatives that are expressly contemplated include alcohol fixation (e.g., methanol fixation, ethanol fixation), glutaraldehyde fixation and paraformaldehyde fixation.

In some embodiments, a tissue sample will be treated to remove embedding material (e.g., to remove paraffin or formalin) from the sample prior to release, capture or modification of nucleic acids. This can be achieved by contacting the sample with an appropriate solvent (e.g., xylene and ethanol washes).

A particularly relevant source for a tissue sample is a mammal. The sample can be derived from an organ, including for example, an organ of the central nervous system such as brain, brainstem, cerebellum, spinal cord, cranial nerve, or spinal nerve; an organ of the musculoskeletal system such as muscle, bone, tendon or ligament; an organ of the digestive system such as salivary gland, pharynx, esophagus, stomach, small intestine, large intestine, liver, gallbladder or pancreas; an organ of the respiratory system such as larynx, trachea, bronchi, lungs or diaphragm; an organ of the urinary system such as kidney, ureter, bladder or urethra; a reproductive organ such as ovary, fallopian tube, uterus, vagina, placenta, testicle, epididymis, vas deferens, seminal vesicle, prostate, penis or scrotum; an organ of the endocrine system such as pituitary gland, pineal gland, thyroid gland, parathyroid gland, or adrenal gland; an organ of the circulatory system such as heart, artery, vein or capillary; an organ of the lymphatic system such as lymphatic vessel, lymph node, bone marrow, thymus or spleen; a sensory organ such as eye, ear, nose, or tongue; or an organ of the integument such as skin, subcutaneous tissue or mammary gland. In some embodiments, a tissue sample is obtained from a bodily fluid or excreta such as blood, lymph, tears, sweat, saliva, semen, vaginal secretion, ear wax, fecal matter or urine.

A sample from a mammal can be considered (or suspected) healthy or diseased when used. In some cases, two samples can be used: a first being considered diseased and a second being considered as healthy (e.g., for use as a healthy control). Any of a variety of conditions can be evaluated, including but not limited to, cancer, an autoimmune disease, cystic fibrosis, aneuploidy, pathogenic infection, psychological condition, hepatitis, diabetes, sexually transmitted disease, heart disease, stroke, cardiovascular disease, multiple sclerosis or muscular dystrophy. Certain contemplated conditions include genetic conditions or conditions associated with pathogens having identifiable mRNA transcript signatures.

### Permeabilizing Agents

Certain embodiments of the instant disclosure feature permeabilizing agents, examples of which tend to compromise and/or remove the protective boundary of lipids often surrounding cellular macromolecules. Disruption of cellular lipid barriers via administration of a permeabilizing agent can provide enhanced physical access to cellular macromolecules, such as DNA, RNA, or proteins, that might otherwise be relatively inaccessible. Specifically contemplated examples of permeabilizing agents include, without limitation: Triton X-100, NP-40, methanol, acetone, Tween 20, saponin, Leucoperm™, and digitonin, among others.

### Nucleic Acid Probe Annealing, Amplification and Sequencing of Target Nucleic Acids

Certain embodiments of the instant disclosure feature nucleic acid primers or probes that are designed to anneal target nucleic acids in or associated with a contacted tissue. A primer is a short nucleic acid sequence that provides a starting point for DNA synthesis. In some embodiments, nucleic acid primers are tagged with barcodes or unique molecular identifiers (UMIs). A “barcode sequence” is a series of nucleotides in a nucleic acid that can be used to identify the nucleic acid, a characteristic of the nucleic acid, or a manipulation that has been carried out on the nucleic acid. In some embodiments the barcode is known as a unique molecular identifier (UMI). The barcode sequence can be a naturally occurring sequence or a sequence that does not occur naturally in the organism from which the barcoded nucleic acid was obtained. A barcode sequence can be unique to a single nucleic acid species in a population, or a barcode sequence can be shared by several different nucleic acid species in a population. By way of further example, each nucleic acid probe in a population can include different barcode sequences from all other nucleic acid probes in the population. Alternatively, each nucleic acid probe in a population can include different barcode sequences from some or most other nucleic acid probes in a population. For example, each probe in a population can have a barcode that is present for several different probes in the population even though the probes with the common barcode differ from each other at other sequence regions along their length. In particular embodiments, one or more barcode sequences that are used with a biological specimen (e.g., a tissue sample) are not present in the genome, transcriptome or other nucleic acids of the biological specimen. For example, barcode sequences can have less than 80%, 70%, 60%, 50% or 40% sequence identity to the nucleic acid sequences in a particular biological specimen.

A nucleic acid probe hybridizes to single-stranded nucleic acid (DNA or RNA) whose base sequence allows probe-target base pairing due to complementarity between the probe and target. The labeled probe is first denatured into single stranded DNA (ssDNA) and then hybridized to the target ssDNA or ssRNA immobilized in situ, e.g., in a matrix or other solid support. The probe is tagged or “labeled” to detect hybridization of the probe to its target sequence. In some embodiments, fluorescent hybridization probes may be used to detect and localize DNA and/or RNA sequences to define the spatial-temporal patterns of gene expression within cells and tissues. In some embodiments, the probe may be a poly-T probe for binding a population of mRNAs and detecting mRNA levels within an annealed population of target mRNA molecules.

In some embodiments, attachment of a nucleic acid probe is non-specific with regard to any sequence differences between the nucleic acid probe and other nucleic acid probes that are or will be attached to a matrix. For example, different probes can have a universal sequence that complements matrix-attached primers, or the different probes can have a common moiety that mediates attachment to the matrix. Alternatively, each of the different probes (or a subpopulation of different probes) can have a unique (or sufficiently unique) sequence that complements a unique (or sufficiently unique) primer bound to the matrix or they can have a unique (or sufficiently unique) moiety that interacts with one or more different reactive moieties in the matrix. In such cases, the unique (or sufficiently unique) primers or unique (or sufficiently unique) moieties can, optionally, be attached at predefined locations in order to selectively capture particular probes, or particular types of probes, at the respective predefined locations.

Nucleic acid probes that are used in a method set forth herein or present in an apparatus or composition of the present disclosure can include barcode sequences, and for embodiments that include a plurality of different nucleic acid probes, each of the probes can include a different barcode sequence from other probes in the plurality. Barcode sequences can be any of a variety of lengths.

Longer sequences can generally accommodate a larger number and variety of barcodes for a population. Generally, all probes in a plurality will have the same length barcode (albeit with different sequences), but it is also possible to use different length barcodes for different probes. A barcode sequence can be at least 2, 4, 6, 8, 10, 12, 15, 20 or more nucleotides in length. Alternatively, or additionally, the length of the barcode sequence can be at most 20, 15, 12, 10, 8, 6, 4 or fewer nucleotides. Examples of barcode sequences that can be used are set forth, for example, in U.S. Patent Application Publication No. 2014/0342921 and U.S. Pat. No. 8,460,865, each of which is incorporated herein by reference.

Sequencing techniques, such as sequencing-by-synthesis (SBS) techniques, are a useful method for determining barcode sequences in situ. SBS can be carried out as follows. To initiate a first SBS cycle, one or more labeled nucleotides, DNA polymerase, SBS primers etc., can be contacted with one or more features in a tissue or cell (e.g., feature(s) where nucleic acid probes are attached to a matrix). Those features where SBS primer extension causes a labeled nucleotide to be incorporated can be detected. Optionally, the nucleotides can include a reversible termination moiety that terminates further primer extension once a nucleotide has been added to the SBS primer. For example, a nucleotide analog having a reversible terminator moiety can be added to a primer such that subsequent extension cannot occur until a deblocking agent is delivered to remove the moiety. Thus, for embodiments that use reversible termination, a deblocking reagent can be delivered to the matrix (before or after detection occurs). Washes can be carried out between the various delivery steps. The cycle can then be repeated n times to extend the primer by n nucleotides, thereby detecting a sequence of length n. Exemplary SBS procedures, fluidic systems and detection platforms that can be readily adapted for use with a composition, apparatus or method of the present disclosure are described, for example, in Bentley et al., Nature 456:53-59 (2008), International Patent Publication Nos. WO 91/06678, WO 04/018497 or WO 07/123744; U.S. Pat. Nos. 7,057,026, 7,329,492, 7,211,414, 7,315,019 or 7,405,281, and U.S. Patent Application Publication No. 2008/0108082, each of which is incorporated herein by reference.

Other sequencing procedures, wherein in some embodiments, the PONIs are released from the tissue include the use of cyclic reactions, such as pyrosequencing. Pyrosequencing detects the release of inorganic pyrophosphate (PPi) as particular nucleotides are incorporated into a nascent nucleic acid strand (Ronaghi, et al., Analytical Biochemistry 242 (1), 84-9 (1996); Ronaghi, Genome Res. 1 1 (1), 3-1 1 (2001); Ronaghi et al. Science 281 (5375), 363 (1998); or U.S. Pat. Nos. 6,210,891, 6,258,568 or 6,274,320, each of which is incorporated herein by reference). In pyrosequencing, released PPi can be detected by being immediately converted to adenosine triphosphate (ATP) by ATP sulfurylase, and the level of ATP generated can be detected via luciferase-produced photons. Thus, the sequencing reaction can be monitored via a luminescence detection system.

Excitation radiation sources used for fluorescence-based detection systems are not necessary for pyrosequencing procedures. Useful fluidic systems, detectors and procedures that can be used for application of pyrosequencing to apparatus, compositions or methods of the present disclosure are described, for example, in International Patent Publication No. WO2012/058096, U.S. Patent Application Publication No. 2005/0191698 A1, or U.S. Pat. No. 7,595,883 or U.S. Pat. No. 7,244,559, each of which is incorporated herein by reference.

Sequencing-by-ligation reactions are also useful, wherein in some embodiments PONIs are released from the tissue, including, for example, those described in Shendure et al. Science 309:1728-1732 (2005); or U.S. Pat. No. 5,599,675 or U.S. Pat. No. 5,750,341, each of which is incorporated herein by reference. Some embodiments can include sequencing-by-hybridization procedures as described, for example, in Bains et al., Journal of Theoretical Biology 135 (3), 303-7 (1988); Drmanac et al., Nature Biotechnology 16, 54-58 (1998); Fodor et al., Science 251 (4995), 767-773 (1995); or International Patent Publication No. WO 1989/10977, each of which is incorporated herein by reference. In both sequencing-by-ligation and sequencing-by-hybridization procedures, target nucleic acids (or amplicons thereof) that are present at sites of an array are subjected to repeated cycles of oligonucleotide delivery and detection. Compositions, apparatus or methods set forth herein or in references cited herein can be readily adapted for sequencing-by-ligation or sequencing-by-hybridization procedures. Typically, the oligonucleotides are fluorescently labeled and can be detected using fluorescence detectors similar to those described with regard to SBS procedures herein or in references cited herein.

Some sequencing embodiments wherein PONIs are released from the tissue, can utilize methods involving the real-time monitoring of DNA polymerase activity. For example, nucleotide incorporations can be detected through fluorescence resonance energy transfer (FRET) interactions between a fluorophore-bearing polymerase and γ-phosphate-labeled nucleotides, or with zeromode waveguides (ZMWs). Techniques and reagents for FRET-based sequencing are described, for example, in Levene et al. Science 299, 682-686 (2003); Lundquist et al. Opt. Lett. 33, 1026-1028 (2008); and Korlach et al. Proc. Natl. Acad. Sci. USA 105, 1 176-1 181 (2008), each of which is incorporated herein by reference.

Some sequencing embodiments, wherein PONIs are released from the tissue, include detection of a proton released upon incorporation of a nucleotide into an extension product. For example, sequencing based on detection of released protons can use an electrical detector and associated techniques that are commercially available from Ion Torrent (Guilford, C T, a Life Technologies and Thermo Fisher subsidiary) or sequencing methods and systems described in U.S. Patent Application Publication Nos. 2009/0026082 A1; 2009/0127589 A1; 2010/0137143 A1; or U.S. Patent Application Publication No. 2010/0282617 A1, each of which is incorporated herein by reference.

Nucleic acid hybridization techniques are also useful methods for determining barcodes both in situ and ex situ. In some embodiments, methods utilize labelled nucleic acid decoder probes that are complementary to at least a portion of a barcode sequence. In some cases, pools of many different probes with distinguishable labels are used, thereby allowing a multiplex decoding operation. The number of different barcodes determined in a decoding operation can exceed the number of labels used for the decoding operation. For example, decoding can be carried out in several stages where each stage constitutes hybridization with a different pool of decoder probes. The same decoder probes can be present in different pools but the label that is present on each decoder probe can differ from pool to pool (i.e., each decoder probe is in a different “state” when in different pools).

Various combinations of these states and stages can be used to expand the number of barcodes that can be decoded well beyond the number of distinct labels available for decoding. Such combinatorial methods are set forth in further detail in U.S. Pat. No. 8,460,865 or Gunderson et al., Genome Research 14:870-877 (2004), each of which is incorporated herein by reference.

A method of the present disclosure can include a step of contacting a biological specimen (i.e., a sectioned tissue sample in which nucleic acid sequence targets of interest have been amplified through bridge amplification, wherein PONIs are formed) with a matrix that has nucleic acid probes attached thereto, as described in PCT/US19/30194. In some embodiments, the nucleic acid probes are randomly located on a matrix. The identity and location of the nucleic acid probes may have been decoded prior to contacting the biological specimen with the matrix.

A nucleic acid probe used in a composition or method set forth herein can include a target capture moiety. In particular embodiments, the target capture moiety is a target capture sequence. The target capture sequence is generally complementary to a target sequence such that target capture occurs by formation of a probe-target hybrid complex. A target capture sequence can be any of a variety of lengths including, for example, lengths exemplified herein in the context of barcode sequences.

In certain embodiments, a plurality of different nucleic acid probes can include different target capture sequences that hybridize to different target nucleic acid sequences from a biological specimen. Different target capture sequences can be used to selectively bind to one or more desired target

All or part of a target nucleic acid that is hybridized to a nucleic acid probe can be copied by extension. For example, an extended probe can include at least, 1, 2, 5, 10, 25, 50, 100, 200, 500, 1000 or more nucleotides that are copied from a target nucleic acid. The length of the extension product can be controlled, for example, using reversibly terminated nucleotides in the extension reaction and running a limited number of extension cycles. The cycles can be run as exemplified for SBS techniques and the use of labeled nucleotides is not necessary.

Modified nucleic acid probes (e.g., extended nucleic acid probes) that are released from an in situ matrix can be pooled to form a fluidic mixture. The mixture can include, for example, at least 10, 100, 1×10³, 1×10⁴, 1×10⁵, 1×10⁶, 1×10⁷, 1×10⁸, 1×10⁹ or more different modified probes. Alternatively or additionally, a fluidic mixture can include at most 1×10⁹, 1×10⁸, 1×10⁷, 1×10⁶, 1×10⁵, 1×10⁴, 1×10³, 100, 10 or fewer different modified probes. The fluidic mixture can be manipulated to allow detection of the modified nucleic acid probes. For example, the modified nucleic acid probes can be separated spatially on a second solid support (i.e., different from the in situ matrix from which the nucleic acid probes were released after having been contacted with a biological specimen and modified), or the probes can be separated temporally in a fluid stream.

Modified nucleic acid probes (e.g., extended nucleic acid probes) can be separated on a bead or other solid support in a capture or detection method commonly employed for microarray-based techniques or nucleic acid sequencing techniques such as those set forth previously. For example, modified probes can be attached to a microarray by hybridization to complementary nucleic acids. The modified probes can be attached to beads or to a flow cell surface and optionally undergo additional rounds of amplification as is carried out in many nucleic acid sequencing platforms. Modified probes can be separated in a fluid stream using a microfluidic device, droplet manipulation device, or flow cytometer. Typically, detection is carried out on these separation devices, but detection is not necessary in all embodiments.

It is further expressly contemplated that in addition to the herein-described sequence features, oligonucleotides of the instant disclosure can possess any number of other art-recognized features while remaining within the scope of the instant disclosure.

In situ Sequencing

In certain aspects of the disclosure, in situ sequencing is performed by any art-recognized mode of parallel (optionally massively parallel) in situ sequencing, examples of which particularly include the previously described SOLID™ method, which is a sequencing-by-ligation technique that can be performed in situ upon a solid support (refer, e.g., to Voelkerding et al, Clinical Chem., 55-641-658, 2009; U.S. Pat. Nos. 5,912,148; and 6,130,073, which are incorporated herein by reference in their entireties). In certain embodiments of the instant disclosure, such sequencing can be performed upon a PONI array in an in situ matrix present on a standard microscope slide, optionally using a standard microscope fitted with sufficient computing power to track and associate individual sequences during progressive rounds of detection, with their spatial position(s). Custom fluidics, incubation times, enzymatic mixes and imaging setup can also be used in performing in situ sequencing.

### “Slide-Seq” Arrays

In certain embodiments, it is expressly contemplated that target nucleic acids and/or amplicons thereof can not only be identified and resolved via performance of in situ methods such as in situ sequencing, but can also be identified and resolved using approaches that retain spatial information of contacted surfaces (e.g., tissues and/or the in situ matrix of the current disclosure) via use of tagged arrays that retain sequence information while NGS sequencing is performed. An exemplary such approach that can readily be used in association with the currently disclosed compositions and methods is the “Slide-Seq” approach of PCT/US19/30194, which enabled RNA capture from tissue with high resolution. In an exemplary application, a matrix of the current disclosure having probe-attached target nucleic acids and/or amplicons (e.g., obtained from a tissue) can be contacted with a “Slide-Seq” array (i.e., a slide-attached bead array with known and/or resolvable spatial tags) and NGS sequencing can be performed upon the target nucleic acids and/or amplicons that have transferred to the “Slide-Seq” array. Using such a combination of methods, the high throughput advantages of NGS sequencing can be applied to the compositions and methods of the instant disclosure, while retaining high resolution spatial information.

PONI—In situ Matrix Components and Preparation

PONI matrices can be formed from any of a variety of matrix-forming monomers or polymers known in the art. Exemplary matrices include a monomer or linear component and a branched component (crosslinking agent), though matrices that include only branch-forming components are also known in the art and can be employed herein. In certain embodiments, the in situ matrix is suitable for providing a scaffold for enzymatic reactions. In some embodiments the in situ matrix is both porous and with sufficient structural integrity to covalently bind nucleic acids, e.g., primers or other molecules of interest, while retaining a level of spatial positioning sufficient to allow for spatial positioning of matrix-associated reactions to be obtained at some level of resolution (e.g., 100 μm or less, or other appropriate value of spatial resolution). In some embodiments, a matrix-associated enzymatic reaction is nucleic acid amplification. In some embodiments, the matrix is cross-linked to a preferred degree (optionally based upon the amount of input crosslinking agent and/or initiator compositions, crosslinking catalysts, or other components). In some embodiments, the monomer or linear polymer is acrylamide, methacrylate, polyethylene glycol (PEG), carboxymethyl cellulose (CMC), polyvinylpyrrolidone (PVP), isopropylacrylamide, hyaluronic acid, heparin, PLA (polylactic acid), PGA (polyglycolide), and PLGA (poly(lactic-co-glycolic acid)), PHA (Polyhydroxyalkanoates), PPF (propylene fumarate), agarose, alginate, chitosan, or ethylene glycol-decorated polyisocyanide (PIC) polymers, derivatives thereof, and combinations thereof. In some embodiments, the cross-linking agent is polyethylene glycol dimethacrylate (optionally triethylene glycol dimethyacrylate (TEGDMA) or tetra(ethylene glycol) dimethacrylate), N,N′-methylene bisacrylamide, trisacrylamide, tetracrylamide, or amine end-functionalized 4-arm star-PEG, derivatives thereof, or combinations thereof. It is also contemplated that sufficiently rigid yet porous matrices for purpose of the instant disclosure can be formed from individual monomers or polymers of any of the preceding monomers or polymers, or by individual polymerizable/cross-linkable components known in the art. In some embodiments, a matrix of the instant disclosure can be polymerized via incubation at a temperature of 4° C. or 37° C., optionally at 4° C. and then 37° C., optionally repeating the temperature incubation steps 1, 2, 3, 4, or 5 times, optionally adding an initiator composition, optionally where the initiator composition is ammonium persulfate (APS) and tetramethylethylenediamine (TEMED), optionally where the initiator composition is riboflavin and TEMED.

In some embodiments, the ratio of the cross-linking agent to the first monomer or linear polymer is at most 1:50 by weight, in some embodiments, the ratio of the cross-linking agent to the monomer or linear polymer is at most 1:100 by weight, in some embodiments, the ratio of the cross-linking agent to the monomer or linear polymer is at most 1:500 by weight, in some embodiments, the ratio of the cross-linking agent to the monomer or linear polymer is at most 1:1,000 by weight, in some embodiments, the ratio of the cross-linking agent to the monomer or linear polymer is at most 1:2,000 by weight, in some embodiments, the ratio of the cross-linking agent to the monomer or linear polymer is at most 1:3,000 by weight, in some embodiments, the ratio of the cross-linking agent to the monomer or linear polymer is at most 1:5,000 by weight, in some embodiments, the ratio of the cross-linking agent to the monomer or linear polymer is at most 1:10,000 by weight, in some embodiments, the ratio of the cross-linking agent to the monomer or linear polymer is at most 1:20,000 by weight, in some embodiments, the ratio of the cross-linking agent to the monomer or linear polymer is at most 1:30,000 by weight, in some embodiments, the ratio of the cross-linking agent to the monomer or linear polymer is at most 1:40,000 by weight, in some embodiments, the ratio of the cross-linking agent to the monomer or linear polymer is at most 1:50,000 by weight, in some embodiments, the ratio of the cross-linking agent to the monomer or linear polymer is at most 1:75,000 by weight, in some embodiments, the ratio of the cross-linking agent to the monomer or linear polymer is at most 1:100,000 by weight, in some embodiments, the ratio of the cross-linking agent to the monomer or linear polymer is at most 1:200,000 by weight, in some embodiments, the ratio of the cross-linking agent to the monomer or linear polymer is at most 1:300,000 by weight, in some embodiments, the ratio of the cross-linking agent to the monomer or linear polymer is at most 1:400,000 by weight, in some embodiments, the ratio of the cross-linking agent to the monomer or linear polymer is at most 1:500,000 by weight, in some embodiments, ratio of the cross-linking agent to the monomer or linear polymer is at most 1:600,000 by weight, in some embodiments, the ratio of the cross-linking agent to the to the monomer or linear polymer is at most 1:700,000 by weight, in some embodiments, the cross-linking agent to the to the monomer or linear polymer is at most 1:800,000 by weight, in some embodiments, the ratio of the cross-linking agent to the to the monomer or linear polymer is at most 1:900,000 by weight, in some embodiments, the ratio of the cross-linking agent to the to the monomer or linear polymer is at most 1:1,000,000 by weight.

### PONI Matrix-Associated Nucleic Acid Primers and Probes

The PONI process features matrix-associated nucleic acid primers or probes, which are used for capture of target nucleic acids, and optionally for amplification in situ. Association of a nucleic acid primer or probe with a matrix component and/or matrix can be performed by art-recognized means, the most common of which employ modified nucleic acid primers or probes to achieve such associations. Exemplary nucleic acid modifications that can be employed to attach a nucleic acid primer or probe to a matrix component and/or matrix include, without limitation, acrydite, biotin-streptavidin, magnetic beads, digoxigenin (DIG), PEG, nanoparticles, peptides, antigens for the purpose of binding an antibody, and related molecules that allow for the initial binding and subsequent polymerization of nucleic acids. In some embodiments, a nucleic acid modification comprising free COOH groups can be activated to become reactive to amine functional groups in a matrix, and vice versa. In some cases, an acrydite moiety can refer to an acrydite analogue generated from the reaction of acrydite with one or more species, such as, for example, the reaction of acrydite with other monomers and cross-linkers during a polymerization reaction. Acrydite moieties may be modified to form chemical bonds with a species to be attached, such as an oligonucleotide. For example, acrydite moieties may be modified with thiol groups capable of forming a disulfide bond or may be modified with groups already having a disulfide bond. The thiol or disulfide may be used as an anchor point for a species to be attached or another part of the acrydite moiety may be used for attachment. In some cases, attachment is reversible, such that when the disulfide bond is broken (e.g., in the presence of a reducing agent), the agent is released from the matrix or other support. In other cases, an acrydite moiety includes a reactive hydroxyl group that may be used for attachment.

### Other Sequencing Methods

Some of the methods and compositions provided herein employ methods of sequencing nucleic acids. A number of DNA sequencing techniques are known in the art, including fluorescence-based sequencing methodologies (see, e.g., Birren et al, Genome Analysis Analyzing DNA, 1, Cold Spring Harbor, N.Y., which is incorporated herein by reference in its entirety). In some embodiments, automated sequencing techniques understood in that art are utilized. In some embodiments, parallel sequencing of partitioned amplicons can be utilized (International Patent Publication No WO2006084132, which is incorporated herein by reference in its entirety). In some embodiments, DNA sequencing is achieved by parallel oligonucleotide extension (see, e.g., U.S. Pat. Nos. 5,750,341; 6,306,597, which are incorporated herein by reference in their entireties). Additional examples of sequencing techniques include the Church polony technology (Mitra et al, 2003, Analytical Biochemistry 320, 55-65; Shendure et al, 2005 Science 309, 1728-1732; U.S. Pat. Nos. 6,432,360, 6,485,944, 6,511,803, which are incorporated by reference), the 454 picotiter pyrosequencing technology (Margulies et al, 2005 Nature 437, 376-380; U.S. Patent Application Publication No. US20050130173, which are incorporated herein by reference in their entireties), the Solexa single base addition technology (Bennett et al, 2005, Pharmacogenomics, 6, 373-382; U.S. Pat. Nos. 6,787,308; 6,833,246, which are incorporated herein by reference in their entireties), the Lynx massively parallel signature sequencing technology (Brenner et al. (2000). Nat. Biotechnol. 18:630-634; U.S. Pat. Nos. 5,695,934; 5,714,330, which are incorporated herein by reference in their entireties), and the Adessi PCR colony technology (Adessi et al. (2000). Nucleic Acid Res. 28, E87; International Patent Publication No. WO 00018957, which are incorporated herein by reference in their entireties).

Next-generation sequencing (NGS) methods can be employed in certain aspects of the instant disclosure to obtain a high volume of sequence information (such as are particularly required to perform deep sequencing of mRNA generated PONIs in a highly efficient and cost effective manner. NGS methods share the common feature of massively parallel, high-throughput strategies, with the goal of lower costs in comparison to older sequencing methods (see, e.g., Voelkerding et al, Clinical Chem., 55:641-658, 2009; MacLean et al, Nature Rev. Microbiol, 7-287-296; which are incorporated herein by reference in their entireties). NGS methods can be broadly divided into those that typically use template amplification and those that do not. Amplification-utilizing methods include pyrosequencing commercialized by Roche as the 454 technology platforms (e.g., GS 20 and GS FLX), the Solexa platform commercialized by Illumina, and the Supported Oligonucleotide Ligation and Detection (SOLID™) platform commercialized by Applied Biosystems. Non-amplification approaches, also known as single-molecule sequencing, are exemplified by the HeliScope platform commercialized by Helicos Biosciences, SMRT sequencing commercialized by Pacific Biosciences, and emerging platforms marketed by VisiGen and Oxford Nanopore Technologies Ltd.

In the Solexa/Illumina platform (Voelkerding et al, Clinical Chem., 55-641-658, 2009; MacLean et al, Nature Rev. Microbiol, 7:287-296; U.S. Pat. Nos. 6,833,246; 7,115,400; 6,969,488, which are incorporated herein by reference in their entireties), sequencing data are produced in the form of shorter-length reads. In this method, single-stranded fragmented DNA is end-repaired to generate 5′-phosphorylated blunt ends, followed by Klenow-mediated addition of a single A base to the 3′ end of the fragments. A-addition facilitates addition of T-overhang adaptor oligonucleotides, which are subsequently used to capture the template-adaptor molecules on the surface of a flow cell that is studded with oligonucleotide anchors. The anchor is used as a PCR primer, but because of the length of the template and its proximity to other nearby anchor oligonucleotides, extension by PCR results in the “arching over” of the molecule to hybridize with an adjacent anchor oligonucleotide to form a bridge structure on the surface of the flow cell. These loops of DNA are denatured and cleaved. Forward strands are then sequenced with reversible dye terminators. The sequence of incorporated nucleotides is determined by detection of post-incorporation fluorescence, with each fluorophore and block removed prior to the next cycle of dNTP addition. Sequence read length ranges from 36 nucleotides to over 50 nucleotides, with overall output exceeding 1 billion nucleotide pairs per analytical run.

Sequencing nucleic acid molecules using SOLID technology (Voelkerding et al, Clinical Chem., 55:641-658, 2009; U.S. Pat. Nos. 5,912,148; and 6,130,073, which are incorporated herein by reference in their entireties) can initially involve fragmentation of the template, ligation to oligonucleotide adaptors, and clonal amplification by emulsion PCR.

Following this, templates are immobilized on a derivatized surface of a glass flow-cell, and a primer complementary to the adaptor oligonucleotide is annealed. However, rather than utilizing this primer for 3′ extension, it is instead used to provide a 5′ phosphate group for ligation to interrogation probes containing two probe-specific bases followed by 6 degenerate bases and one of four fluorescent labels. In the SOLID system, interrogation probes have 16 possible combinations of the two bases at the 3′ end of each probe, and one of four fluors at the 5′ end. Fluor color, and thus identity of each probe, corresponds to specified color-space coding schemes. Multiple rounds (usually 7) of probe annealing, ligation, and fluor detection are followed by denaturation, and then a second round of sequencing using a primer that is offset by one base relative to the initial primer. In this manner, the template sequence can be computationally re-constructed, and template bases are interrogated twice, resulting in increased accuracy. Sequence read length averages 35 nucleotides, and overall output exceeds 4 billion bases per sequencing run.

In certain embodiments, nanopore sequencing is employed (see, e.g., Astier et al, J. Am. Chem. Soc. 2006 Feb. 8; 128 (5): 1705-10, which is incorporated by reference). The theory behind nanopore sequencing has to do with what occurs when a nanopore is immersed in a conducting fluid and a potential (voltage) is applied across it. Under these conditions a slight electric current due to conduction of ions through the nanopore can be observed, and the amount of current is exceedingly sensitive to the size of the nanopore. As each base of a nucleic acid passes through the nanopore (or as individual nucleotides pass through the nanopore in the case of exonuclease-based techniques), this causes a change in the magnitude of the current through the nanopore that is distinct for each of the four bases, thereby allowing the sequence of the DNA molecule to be determined.

The Ion Torrent technology is a method of DNA sequencing based on the detection of hydrogen ions that are released during the polymerization of DNA (see, e.g., Science 327 (5970): 1190 (2010); U.S. Patent Application Publication Nos. 20090026082, 20090127589, 20100301398, 20100197507, 20100188073, and 20100137143, which are incorporated herein by reference in their entireties). A microwell contains a template DNA strand to be sequenced. Beneath the layer of microwells is a hypersensitive ISFET ion sensor. All layers are contained within a CMOS semiconductor chip, similar to that used in the electronics industry. When a dNTP is incorporated into the growing complementary strand a hydrogen ion is released, which triggers a hypersensitive ion sensor. If homopolymer repeats are present in the template sequence, multiple dNTP molecules will be incorporated in a single cycle. This leads to a corresponding number of released hydrogens and a proportionally higher electronic signal. This technology differs from other sequencing technologies in that no modified nucleotides or optics are used. The per base accuracy of the Ion Torrent sequencer is approximately 99.6% for 50 base reads, with approximately 100 Mb generated per run. The read-length is 100 base pairs. The accuracy for homopolymer repeats of 5 repeats in length is approximately 98%. The benefits of ion semiconductor sequencing are rapid sequencing speed and low upfront and operating costs.

### Imaging Image Assembly

In certain embodiments, the spatial locations of a large number of amplicons (including barcoded amplicons) within an array can first be assigned to an image location, with all associated nucleic acid sequence data also assigned to that position. High resolution images representing the extent of capture of individual or grouped nucleic acid sequences across the various spatial positions of the in situ matrix can then be generated using the underlying sequence information. Images (i.e., pixel coloring and/or intensities) can be adjusted and/or normalized using any (or any number of) art-recognized technique(s) deemed appropriate by one of ordinary skill in the art.

In certain embodiments, a high-resolution image of the instant disclosure is an image in which discrete features (e.g., pixels) of the image are spaced at 50 μm or less. In some embodiments, the spacing of discrete features within the image is at 40 μm or less, optionally 30 μm or less, optionally 20 μm or less, optionally 15 μm or less, optionally 10 μm or less, optionally 9 μm or less, optionally 8 μm or less, optionally 7 μm or less, optionally 6 μm or less, optionally 5 μm or less, optionally 4 μm or less, optionally 3 μm or less, optionally 2 μm or less, or optionally 1 μm or less.

Images can be obtained using detection devices known in the art. Examples include microscopes configured for light, bright field, dark field, phase contrast, fluorescence, reflection, interference, or confocal imaging. A biological specimen can be stained prior to imaging to provide contrast between different regions or cells. In some embodiments, more than one stain can be used to image different aspects of the specimen (e.g., different regions of a tissue, different cells, specific subcellular components or the like). In other embodiments, a biological specimen can be imaged without staining.

In particular embodiments, a fluorescence microscope (e.g., a confocal fluorescent microscope) can be used to detect a biological specimen that is fluorescent, for example, by virtue of a fluorescent label. Fluorescent specimens can also be imaged using a nucleic acid sequencing device having optics for fluorescent detection such as a Genome Analyzer®, MiSeq®, NextSeq® or HiSeq® platform device commercialized by Illumina, Inc. (San Diego, CA); or a SOLID™ sequencing platform commercialized by Life Technologies (Carlsbad, CA). Other imaging optics that can be used include those that are found in the detection devices described in Bentley et al., Nature 456:53-59 (2008), International Patent Publication Nos. WO 91/06678, WO 04/018497 or WO 07/123744; U.S. Pat. Nos. 7,057,026, 7,329,492, 7,211,414, 7,315,019 or 7,405,281, and U.S. Patent Application Publication No. 2008/0108082, each of which is incorporated herein by reference.

An image of a biological specimen can be obtained at a desired resolution, for example, to distinguish tissues, cells or subcellular components. Accordingly, the resolution can be sufficient to distinguish components of a biological specimen that are separated by at least 0.5 μm, 1 μm, 5 μm, 10 μm, 50 μm, 100 μm, 500 μm, 1 mm or more. Alternatively or additionally, the resolution can be set to distinguish components of a biological specimen that are separated by at least 1 mm, 500 μm, 100 μm, 50 μm, 10 μm, 5 μm, 1 μm, 0.5 μm or less.

### Kits

The instant disclosure also provides kits containing agents of this disclosure for use in the methods of the present disclosure. Kits of the instant disclosure may include one or more containers. In some embodiments, the kits further include instructions for use in accordance with the methods of this disclosure. In some embodiments, these instructions comprise a description of administration of the agent (e.g., protein constructs and nucleic acid constructs of the instantly disclosed system, optionally with or in a viral vector, such as AAV or the like) to assess cellular connectivity and/or to diagnose, e.g., a disease and/or malignancy. In some embodiments, the instructions comprise a description of how to create a tissue cryosection, treat a tissue section with a forward and reverse amplification primers; matrix precursor monomers or linear polymers; a cross-linking agent; a reverse transcriptase; a flow cell to perform bridge amplification and generate polonies in situ (PONIs); sequencing primers and reversible 3′ fluorescent nucleotide blockers to sequence the PONIs by synthesis; and instructions for use. The kit may further comprise a description of selecting an individual suitable for treatment based on identifying whether that subject has a certain pattern of nucleic acid amplification, sequence and/or localization of one or more nucleic acid sequences in a sample.

Instructions supplied in the kits of the instant disclosure are typically written instructions on a label or package insert (e.g., a paper sheet included in the kit), but machine-readable instructions (e.g., instructions carried on a magnetic or optical storage disk) are also acceptable.

The label or package insert indicates that the composition is used for detecting subcellular fusion protein, compartment and/or organelle localization, or for detecting cell-cell connectivity (e.g., across a chemical or electrical synapse). Instructions may be provided for practicing any of the methods described herein.

The kits of this disclosure are in suitable packaging. Suitable packaging includes, but is not limited to, vials, bottles, jars, flexible packaging (e.g., sealed Mylar or plastic bags), and the like. The container may further comprise a pharmaceutically active agent.

Kits may optionally provide additional components such as buffers and interpretive information. Normally, the kit comprises a container and a label or package insert(s) on or associated with the container.

The practice of the present disclosure employs, unless otherwise indicated, conventional techniques of chemistry, molecular biology, microbiology, recombinant DNA, genetics, immunology, cell biology, cell culture and transgenic biology, which are within the skill of the art. see, e.g., Maniatis et al., 1982, Molecular Cloning (Cold Spring Harbor Laboratory Press, Cold Spring Harbor, N.Y.); Sambrook et al., 1989, Molecular Cloning, 2nd Ed. (Cold Spring Harbor Laboratory Press, Cold Spring Harbor, N.Y.); Sambrook and Russell, 2001, Molecular Cloning, 3rd Ed. (Cold Spring Harbor Laboratory Press, Cold Spring Harbor, N.Y.); Ausubel et al., 1992), Current Protocols in Molecular Biology (John Wiley & Sons, including periodic updates); Glover, 1985, DNA Cloning (IRL Press, Oxford); Anand, 1992; Guthrie and Fink, 1991; Harlow and Lane, 1988, Antibodies, (Cold Spring Harbor Laboratory Press, Cold Spring Harbor, N.Y.); Jakoby and Pastan, 1979; Nucleic Acid Hybridization (B. D. Hames & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Methods In Enzymology, Vols. 154 and 155 (Wu et al. eds.), Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); Riott, Essential Immunology, 6th Edition, Blackwell Scientific Publications, Oxford, 1988; Hogan et al., Manipulating the Mouse Embryo, (Cold Spring Harbor Laboratory Press, Cold Spring Harbor, N. Y., 1986); Westerfield, M., The zebrafish book. A guide for the laboratory use of zebrafish (Danio rerio), (4th Ed., Univ. of Oregon Press, Eugene, 2000).

Unless otherwise defined, all technical and scientific terms used herein have the same meaning as commonly understood by one of ordinary skill in the art to which this disclosure belongs. Although methods and materials similar or equivalent to those described herein can be used in the practice or testing of the present disclosure, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Reference will now be made in detail to exemplary embodiments of the disclosure. While the disclosure will be described in conjunction with the exemplary embodiments, it will be understood that it is not intended to limit the disclosure to those embodiments. To the contrary, it is intended to cover alternatives, modifications, and equivalents as may be included within the spirit and scope of the disclosure as defined by the appended claims. Standard techniques well known in the art or the techniques specifically described below were utilized.

### EXAMPLES

### Example 1: Brain-Wide Ascertainment of Projection Neuron Targets

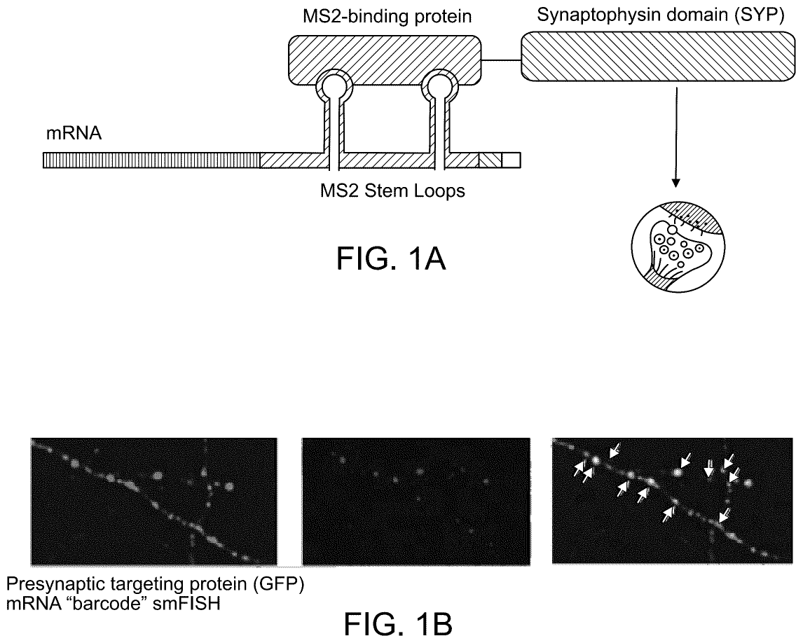

A specific and sensitive toolset was initially developed for trafficking exogenously introduced mRNA to presynaptic compartments of in vitro and in vivo neurons. The general strategy is outlined in FIG. 1A, in which a synaptic trafficking protein and an mRNA reporter were jointly introduced to the same neuron. The trafficking protein is a fusion of a synaptophysin domain, which shuttles to presynaptic boutons, and the PP7 coat protein (PCP), which binds to a specific RNA stem loop motif. The mRNA reporter transcript contains tandem PP7 phage operator stem-loop repeats in the 3′ UTR. When introduced into the same neuron, the stem-loop-containing mRNA reporter transcript binds tightly to the PP7 coat protein (PCP), enabling the RNA to be shuttled to the presynaptic compartment through the natural trafficking of the synaptophysin domain.

To characterize the trafficking of mRNA in neuronal cells, cultured primary cortical neurons were transfected with a specific targeting protein and a specific stem-loop-containing mRNA reporter. Simultaneous single-molecule fluorescence in situ hybridization (smFISH) for the mRNA reporter transcript, and imaging of the fluorescent presynaptic targeting protein demonstrated highly sensitive and specific trafficking of transcripts to presynaptic compartments (FIG. 1B, arrows highlight presynaptic boutons). To confirm the system functioned in the intact CNS, both the synaptic trafficking protein and mRNA reporter were packaged into Adeno-associated viruses (AAVs) and were intracranially delivered to several brain regions. Specifically, a hippocampal subpopulation projecting to the retrosplenial cortex (RSP) was transduced with the present system (FIG. 1C). Extensive presynaptic-specific exogenous mRNA transport was observed in RSP that was entirely dependent on the stem loop interactions with the presynaptic targeting protein (FIG. 1C, right). The efficiency of mRNA labeling of presynaptic sites was 80.8%, and was entirely dependent on the presence of MS2 stem loops (FIG. 1D). In addition, the functionality of the system was confirmed in other circuits such as thalamus to cortex, and CA3 to CA1 in hippocampus. In thalamocortical projection neurons, no decrease in the efficiency of trafficking of mRNA to the presynaptic compartment has been observed. These results demonstrated the sensitivity and specificity of the present system for labeling presynaptic boutons with mRNA both in vivo and in vitro.

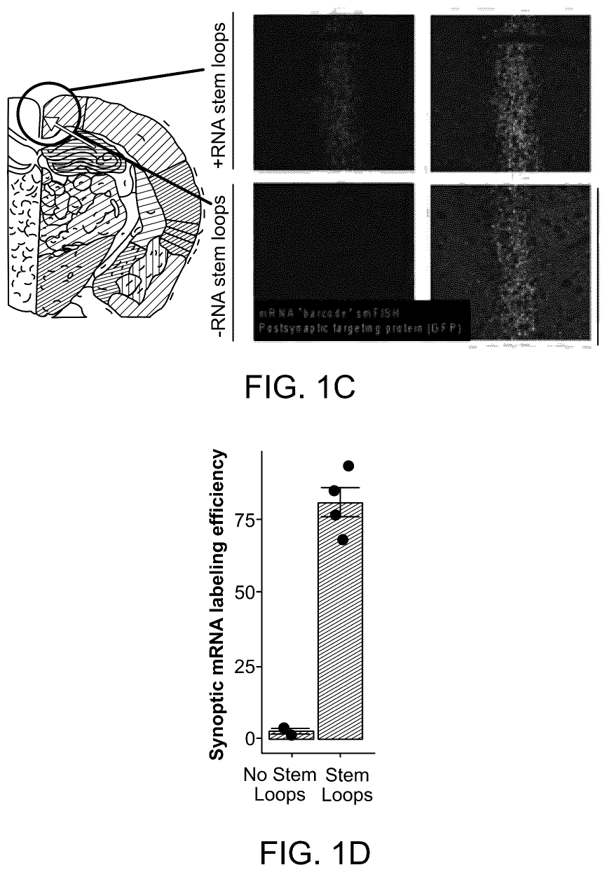

While good presynaptic targeting of the synaptophysin fusion construct was observed in preliminary work (FIGS. 1B-1D), it was also contemplated that an alternative approach could be utilized, if needed, by developing a fibronectin intrabody for binding to presynaptic components (noting that the fibronectin intrabody was described as synaptically inert when targeted to the postsynaptic side). Fibronectin intrabodies generated by RNA display (FingRs) were raised against alpha-Synuclein, which appeared to target presynaptic terminals even more precisely than exogenous synaptophysin (FIGS. 2A-2C), validating the FingR approach. However, alpha-Synuclein is not found at all presynaptic terminals, so it was also contemplated that a FingR targeting the presynaptic scaffolding protein Bassoon could also be developed.

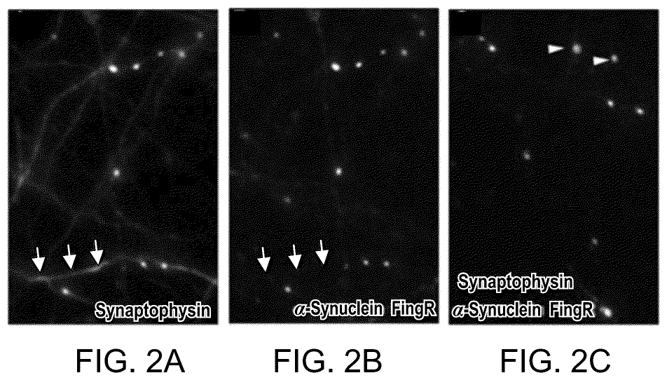

A presynaptically targeted, diversely barcoded AAV virus was generated (FIGS. 3A-3B) and injected into the ventral posteromedial (VPM) nucleus of the thalamus (FIG. 3C). One week later, tissue was harvested and the individual nuclei were dissociated from the VPM injection site, and subjected to high-throughput snRNA-seq (without FACS enrichment). A total of 11,437 cells were sequenced, and such sequencing confirmed that the identification of distinct neuronal subtypes within thalamus was possible (FIGS. 3D-3F), demonstrating how the present mRNA trafficking system does not compromise transcriptional identity of transduced neurons.

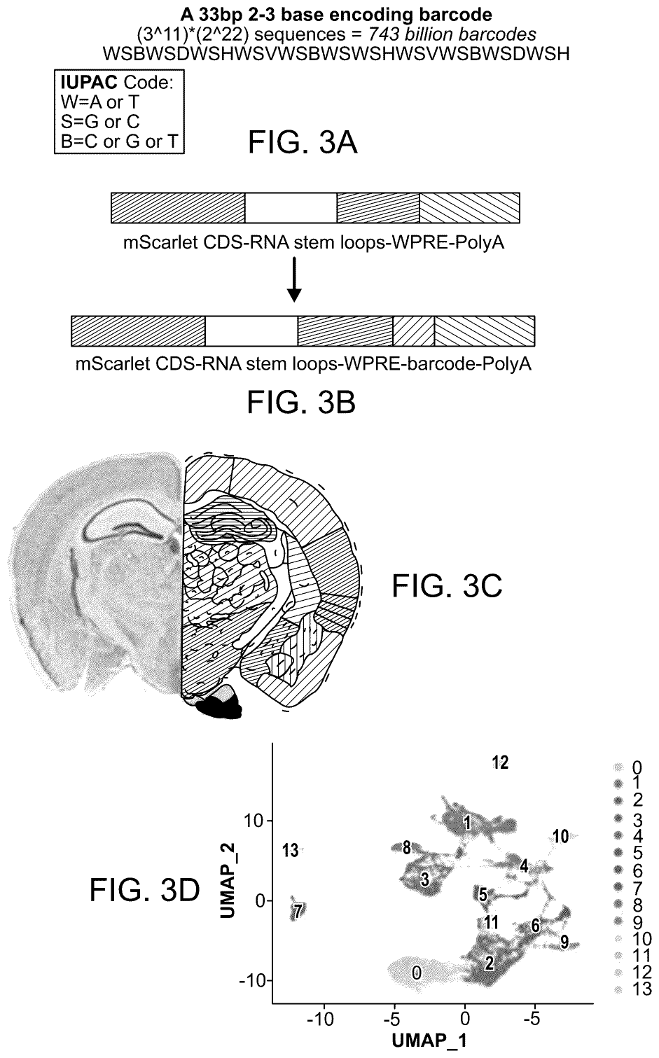

The molecular diversity and distribution of the barcoded virions was also examined. Within the excitatory thalamic principal neurons (FIGS. 3E, 4A), a median of 60 unique barcodes per neuron was detected (FIG. 4B), confirming that many AAV virions enter each neuron. Critically, most barcode sequences (94.4%) were detected in only one cell, demonstrating that the barcoded AAV population was highly diverse and well-balanced (FIG. 4C).

In previous work, a tool for RNA capture from tissue with high resolution, termed “Slide-seq”, was developed (refer to PCT/US19/30194). The Slide-seq approach localized transcriptome-wide gene expression at 10-micron spatial resolution in fresh-frozen mammalian brain tissue sections. The instant disclosure has quantified presynaptic projections by performing snRNA-seq (single cell sequencing) at the injection site, to obtain a white list of barcodes expressed in the projection neurons, and Slide-seq was then performed at the projection targets, to quantify barcode locations at those sites. Intersection of these barcodes enables reconstruction of projections by DNA sequencing.

Slide-seq has several key advantages that make it well-suited to the high-throughput quantification of projection targets:
1 Transcripts are quantified by DNA sequencing, meaning that complex barcodes can be easily parsed (in contrast to hybridization-based strategies for transcript detection).
2. The quantification of transcripts is done ex situ, meaning that the density of transcripts at a given Slide-seq pixel does not affect the detection efficiency. Therefore, neurons with high MOI (and hence many barcodes) can be as easily quantified as neurons that are sparsely infected. This is in contrast to imaging-based strategies for detection, in which molecular crowding mandates sparse, low MOI infection of neurons.
3. The Slide-seq assay is very straightforward to perform, meaning that dozens of 10 micron-thick sections can be assayed in a single experiment.

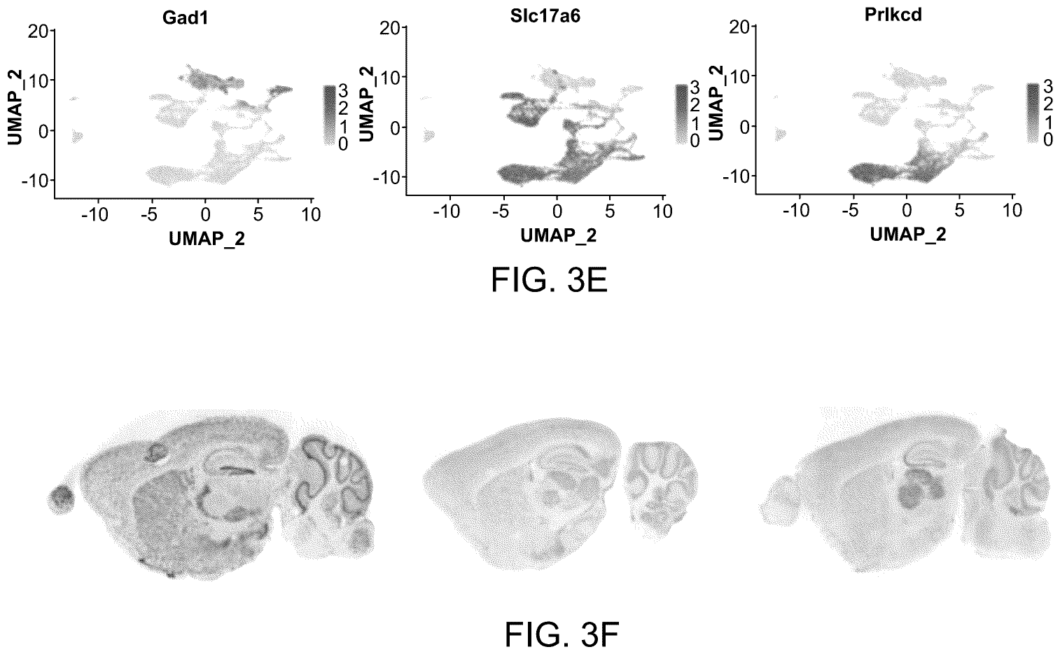

In initial work with the fusion proteins and RNA reporter molecules of the instant disclosure, it was demonstrated that Slide-seq could detect presynaptically transported mRNA. Specifically, the lateral geniculate nucleus (LGN) of thalamus was injected with AAVs carrying the presently-disclosed presynaptic targeting system. A week later, the mice were sacrificed, and fresh-frozen coronal sections were assayed using Slide-seq. A plot of the counts of the exogenous transcript demonstrated strong expression in the LGN (bottom left of array, FIG. 5A), as well as counts in the upper layers of the overlying cortex. The observed distribution of the viral transcript within cortex indicated labeling in the layers known to receive thalamic input (most especially layer 4, marked by Rorb, FIG. 5B). To maximize capture of the exogenous mRNA, a biotinylated primer was spiked into the Slide-seq transcriptome amplification step, to specifically amplify—and then purify by streptividin affinity—the barcoded AAV transcript. Targeted enrichment and deep sequencing enabled the detection of hundreds of barcode transcripts on beads in layer 4 of cortex, while maintaining labeling specificity (FIG. 5C).

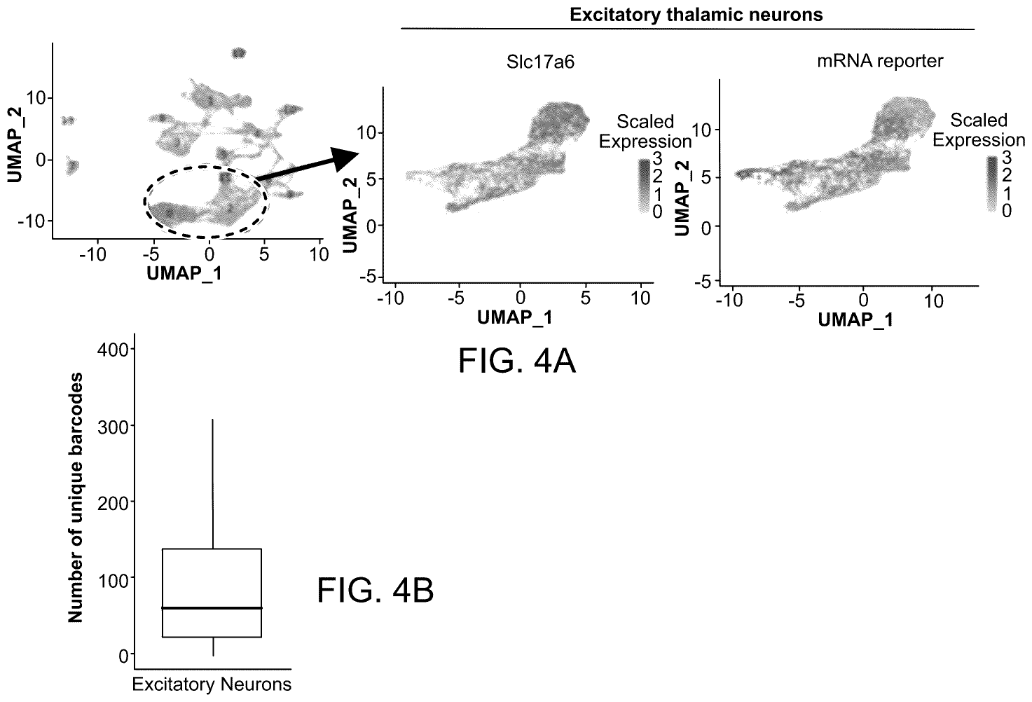

The postsynaptic trafficking systems have been implemented and optimized both in vitro and in vivo (FIG. 6A). Introduction of the present system into primary cortical neurons resulted in robust transport of mRNA to excitatory dendritic spines (FIG. 6B) and an optimized version of the current system labelled 100% postsynaptic compartments (FIG. 6C). To determine its efficacy in vivo, the system was packaged into AAVs and injected into the CA2 region of hippocampus. Only in the presence of stem loops was accumulation of the viral mRNA transcript in the synaptic layers of CA2 (FIG. 6D) possible. The instant disclosure has therefore provided a system to traffic mRNA to inhibitory postsynaptic compartments, which has been validated in vitro (FIG. 6E).

### Example 2: Generation and Validation of a Massively Diverse AAV Barcode Library Targeting the Presynaptic Compartment

A highly diverse DNA sequence was introduced into the 3′ UTR of the current pre-synaptic trafficking system, providing a barcode nucleic acid that could be detected and associated with a specific viral infection event/neuron. FIG. 3A shows the structure of the 33-base pair barcode currently employed, whose theoretical diversity is on the order of 10¹¹ sequences. Critically, this sequence diversity should be sufficient to uniquely barcode each AAV virion that is stereotactically injected into the animal, allowing for the tracking of single-cell-infected viruses in vivo.

### Example 3: Synapse-seq Components and Function

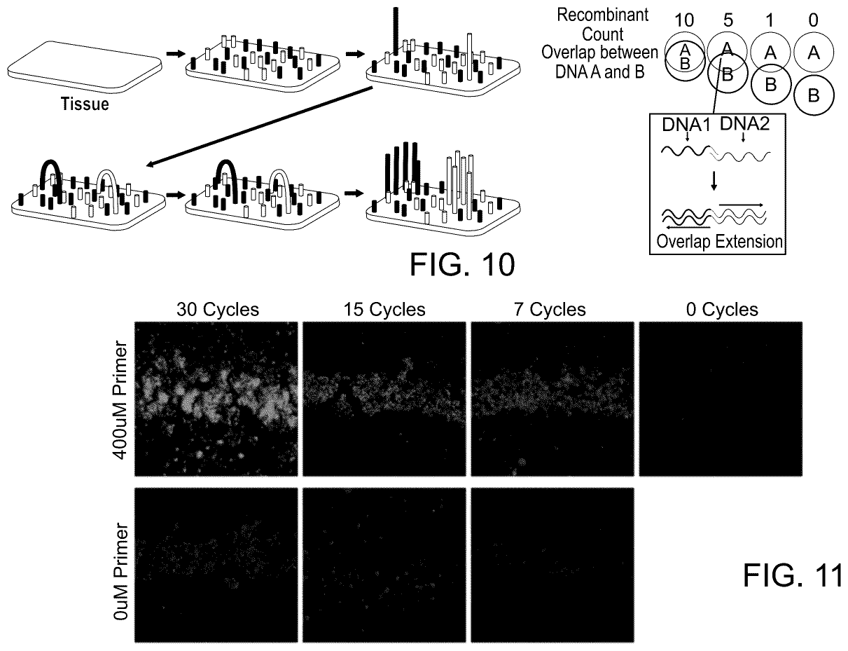

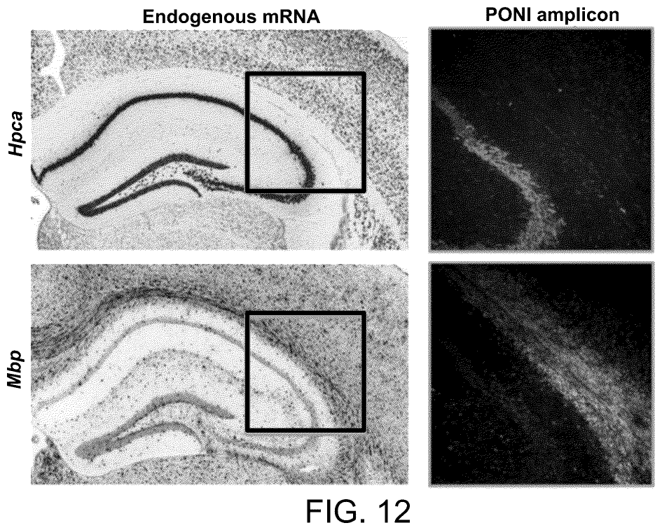

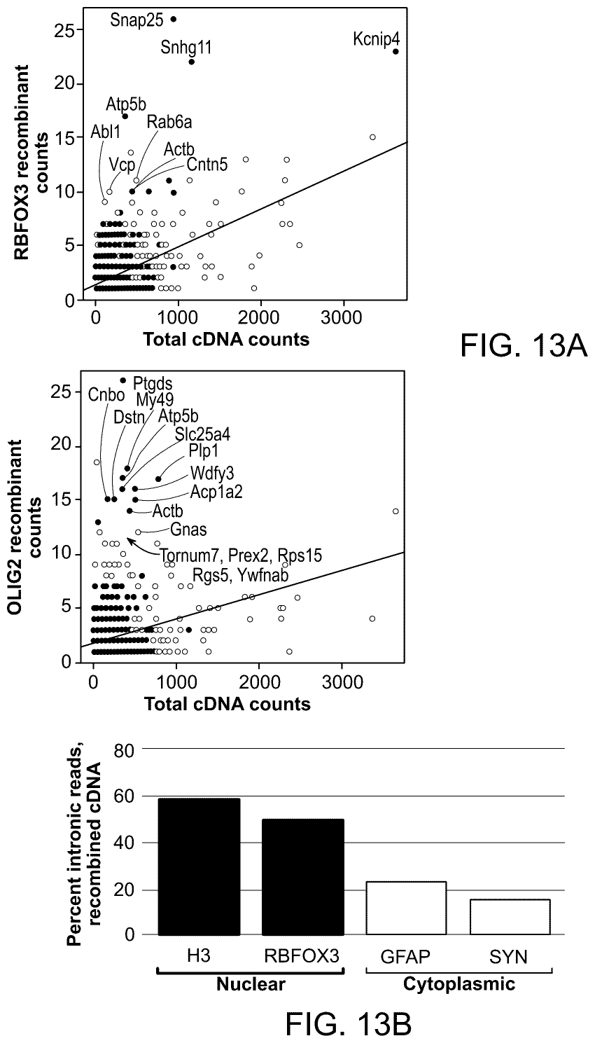

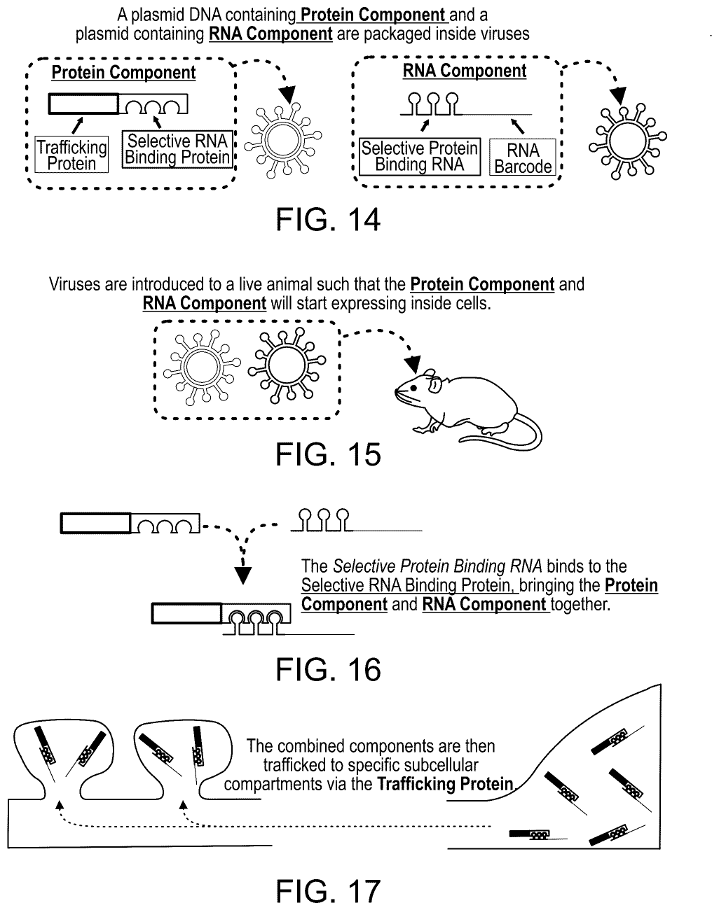

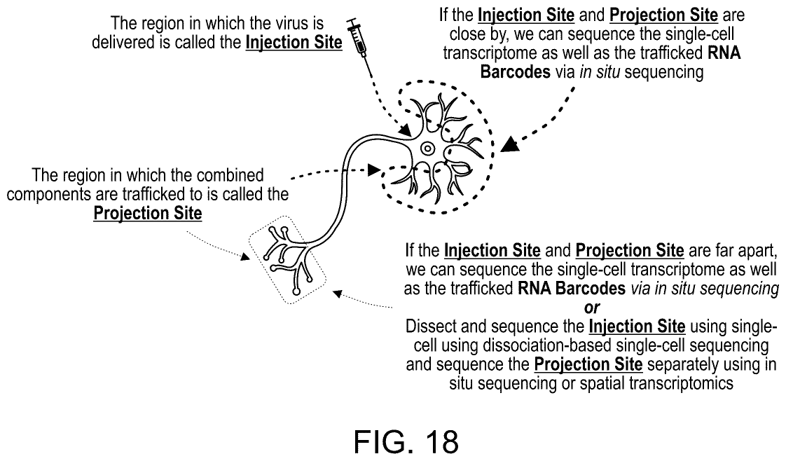

A minimally invasive means of effectively adding a barcode nucleic acid tag to a newly-expressed fusion protein in a living cell and/or to a protein, subcellular compartment, organelle, etc. associated with such a fusion protein, was designed, with the specific goal of applying such a system to detection of neuronal connectivity, which could advantageously be performed in unperturbed or minimally perturbed cells, tissues and/or organisms using such a system. An exemplary “Synapse-seq” system of the instant disclosure provides a plasmid DNA containing a protein component and a plasmid containing an RNA component, packaged inside viruses (e.g., AAV or other non-toxic viral vector). For labeling of synapses, the protein component is a fusion protein containing a trafficking protein (synaptophysin as initially exemplified) fused with a selective RNA binding protein. The RNA component of the current system is a fusion oligoribonucleotide containing a selective protein binding RNA (capable of binding the RNA binding protein of the protein component) and an RNA barcode (FIG. 14). The virally-packaged components can be introduced to a live animal such that the protein component and the RNA component start expressing inside cells (FIG. 15). Upon expression, the RNA binding protein domain of the fusion protein binds the selective protein binding RNA of the oligoribonucleotide, bringing the protein component and the RNA component together (FIG. 16). The combined components are subsequently trafficked to specific subcellular compartments via the trafficking protein (FIG. 17). After a sufficient length of time has passed for trafficking/barcode localization to occur (e.g., 7 days post-infection), detection of axonal barcodes can be performed. Virus is initially delivered to the injection site. The region in which the combined components of the instant system are trafficked to within the virally infected cell is called the projection site, which may be at a distance (e.g., at a synaptic terminus of a neuron having a long axon), or in close proximity to the rest of the virally infected cell body. If the injection site and the projection site are in proximity, the single-cell transcriptome and the trafficked RNA barcodes can both be sequenced via in situ sequencing. If the injection site and the projection site are far apart, the injection site can be dissected and sequenced using single cell dissociation-based single-cell sequencing and the projection site can be sequenced separately using in situ sequencing or spatial transcriptomics. Alternatively, if the injection site and the projection site are far apart, the single-cell transcriptome and the trafficked RNA barcodes can also be sequenced via in situ sequencing (FIG. 18).

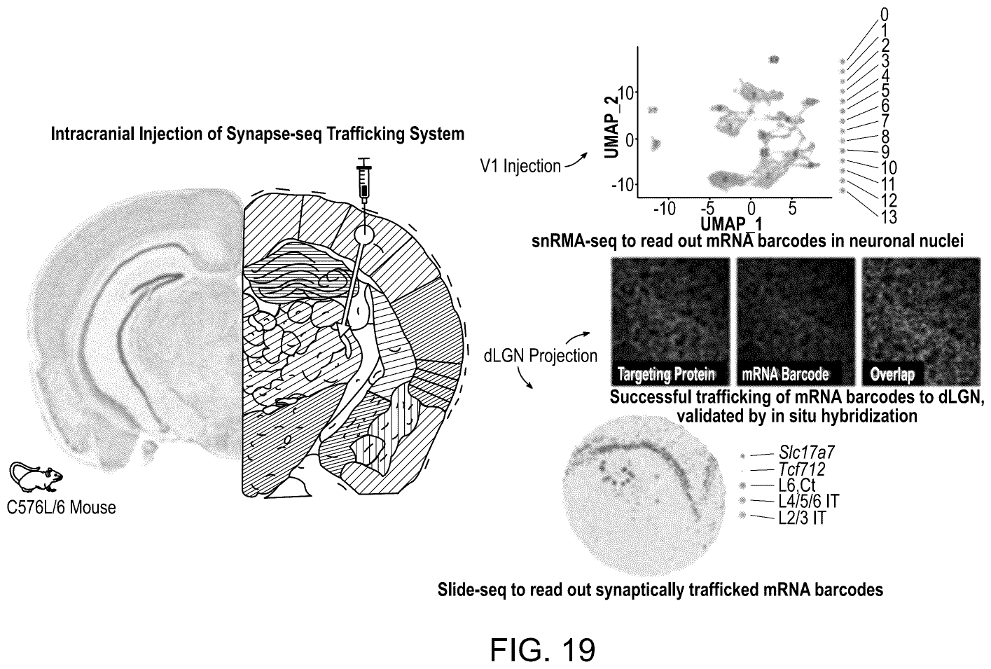

Intracranial injection of the instant Synapse-seq trafficking system was performed. A viral V1 injection of the current system was administered into the dorsolateral geniculate nucleus (dLGN) region of the brain of C57BL/6 mice, located in the thalamus. The results of the V1 injection were graphed in a UMAP presentation, utilizing snRNA-seq to read out mRNA barcodes in neuronal nuclei. There was evidence of successful trafficking of mRNA barcodes to dLGN, validated by in situ hybridization. Successful trafficking of the targeting protein and the associated mRNA barcode was therefore accomplished, and an image of their overlap was generated (FIG. 19). Slide-seq was subsequently used to read out synaptically trafficked mRNA barcodes present in a projection of the dLGN. The barcodes read out were Slc17a7, Tcf712, L6 CT, L4/5/6 IT, and L2/3 IT (Slc17a7 and Tcf712 were the genes plotted to show the structure/location of the brain section, while L6 CT, L4/5/6 IT, L2/3 IT were the cell-types (as identified via snRNA-seq single nucleus sequencing/transcriptome profiling) that were associated with the barcode).

### Example 4: Application of Synapse-seq to Long-Range Corticothalamic Projections

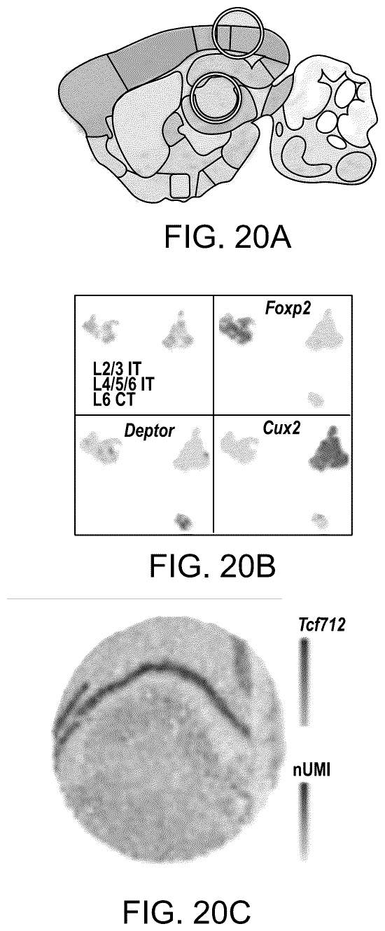

A key question regarding the application of the instant subcellular barcoding system to measure projection connectivity was the extent to which AAV barcodes expressed in the cell's nucleus could be matched to barcodes in the presynaptic processes. To assess this, 50 nL (8×10⁸ viral genomes) of AAV harboring a massively diverse barcode were injected into primary visual cortex (VISP), whose layer 6 corticothalamic (CT) pyramidal neurons send projections to the dorsal lateral geniculate nucleus of the thalamus (dLGN) (FIG. 20A). To molecularly identify infected cells, single-nucleus RNA-seq (snRNA-seq) was performed on the injection site VISP cells. A total of 1,968 cells showed positive expression of the current AAV transcript. These infected cells integrated well with the non-integrated cells (data not shown), demonstrating that AAV expression has a minimal effect on overall transcriptome integrity. Infected cells were clustered by gene expression, annotating individual neuronal clusters (FIG. 20B) as upper or lower intratelencephalic (IT, identified by expression of Cux2 or Deptor respectively, N=1,145 AAV+ cells) and corticothalamic (CT, identified by expression of Foxp2 and the absence of Nxph4, Trhr, or Fam84b, N=823 AAV+ cells).

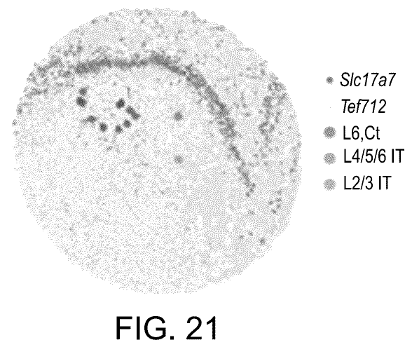

To determine whether projections could be measured with the barcoded AAV, Slide-seq was then performed on three serial coronal 10-micron sections of the dLGN. The Slide-seq transcript capture procedure was optimized for the current system, testing a variety of gentle detergents and chemical modifications to the Slide-seq barcoded oligonucleotide beads, to maximize capture of presynaptic AAV transcripts on the spatial arrays. Expression of Tcf712 inferior to the boundary of the hippocampus enabled identification the dLGN region in the aligned sequence data (FIG. 20C). In total, within the dLGN, 58,926 unique AAV barcodes were detected, of which 24 matched with perfect sequence identity (33 base pair barcode with 10¹¹ sequence diversity) to the 1,968 snRNA-seq profiles. A highly significant majority of these perfectly matched barcodes derived from the CT cluster population (FIG. 21, N=19, ×2=15.42, p<0.0001), demonstrating the tool's ability to map long-range projections in the brain.

The initial pilot experiment profiled only 1,968 VISP nuclei and just three serial sections of dLGN (covering less than 1% of the total volume of this region). However, this approach is highly scalable: a single scientist can easily process 60 Slide-seq arrays and hundreds of thousands of cells per week. Crucially, target region sequencing by Slide-seq is also very cost-efficient (1 million reads required per array), making it practical to sequence hundreds of arrays at once. It is anticipated that hundreds or thousands of projections could be routinely mapped at cell-type-specific resolution using the current instantiation of this technology. Application of the following to this process are also contemplated: 1) FACS-enrichment for infected cells to specifically capture AAV-infected neurons; 2) implementation of network-based barcode edit distance correction approaches to increase the matching rate; 3) Address artefacts of PCR that could swap barcode-cell identity and hence mis-assign barcodes to incorrect single cell clusters—these have been observed in 10× Genomics single-cell data, and several strategies are available to solve this problem that could be computationally adapted to the barcode transcript (e.g., Biorxiv 791699).

### Example 5: Quantification of Postsynaptic Spine Density at Single Cell Resolution, in a Murine Model of Alzheimer's Disease

Utilizing a postsynaptic trafficking system of the instant disclosure, it can be determined which of several potential genomic readouts can properly quantify synaptic density. In the CA1 field of mouse hippocampus, it has been previously demonstrated that, in an amyloidosis model (5XFAD), synapses in the stratum lacunosum moleculare (SLM) are specifically depleted by 12 months of age, whereas synapses in the more proximal stratum radium (SR) are not. By leveraging this known, regionally defined difference in synaptic loss, it is possible to critically assess the performance of different genomic readouts of synaptic density in vivo.

The first and simplest strategy for quantifying synapses is to use bulk purified RNA. Specifically, the barcoded postsynaptic tagging system disclosed herein is transduced by stereotactic injection into the CA1 field of hippocampus. The SLM and SR is individually laser microdissected and RNA purified from each layer and the viral AAV barcodes sequenced. Simultaneously, the granular layer of CA1, containing the cell bodies, is prepared for single nucleus RNA-seq (snRNA-seq). Single cell data is used to build a whitelist of AAV barcodes that have infected CA1 granular cell somas, and these barcodes are matched to those sequenced in the SLM and SR synaptic layers. In the 5xFAD mice, a specific reduction in the digital counts of CA1 barcodes in the SLM is expected, and not in the SR. These data demonstrate the ability of bulk RNA sequencing of synaptic layers to read out changes in synaptic density.

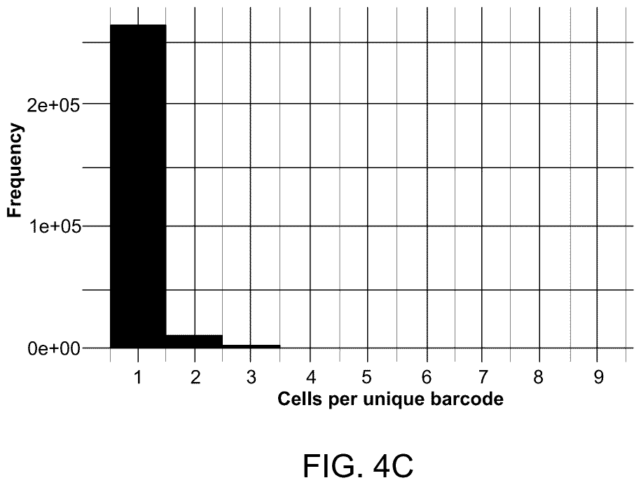

Bulk quantification may not be capable of distinguishing between changes in synaptic number and synaptic bouton size. It also requires that the synaptic layers are spatially separated from the cell bodies to enable microdissection. In an alternative approach, using the same CA1 transduction model, the transduced tissue is dissociated and simultaneously nuclei are isolated for snRNA-seq and biochemically purify synaptosomes. Synaptosomes are subsequently sorted on a FACS to enrich for synapses infected with the postsynaptic trafficking system that expresses fluorescent reporter proteins. mRNA in bulk can be purified from this sorted synaptosome prep, allowing the quantification of counts of AAV barcodes within a fixed number of synaptosomes; or single-synapse RNA sequencing can be performed by loading purified synapses into a microfluidic droplet generator. The foundational technology was developed for performing high-throughput droplet-based single-cell sequencing, and one could easily apply the same device and bead-based barcoding technology to the sequencing of individual synapses using recent massive throughput improvements in droplet generation. In addition, it has been demonstrated that the isolation of synaptosomes that have been labelled with the instant postsynaptic mRNA trafficking system is robust, and it is possible to image examples of pre- and postsynaptic, as well as exclusively post-synaptic, pairs in the instant synaptosome preparations (FIG. 7).

The sequencing of synapses in situ presents a challenge. Synaptosomes may pose several technical challenges: first, they may leak RNA, since ultrastructural analyses suggest some may lack postsynaptic membrane integrity, although protocols have been refined to enrich intact, functional particles. Second, synaptosomes may stick together, may be of different sizes, or may be insufficiently pure, making the barcode quantification a poor proxy for synapse number.

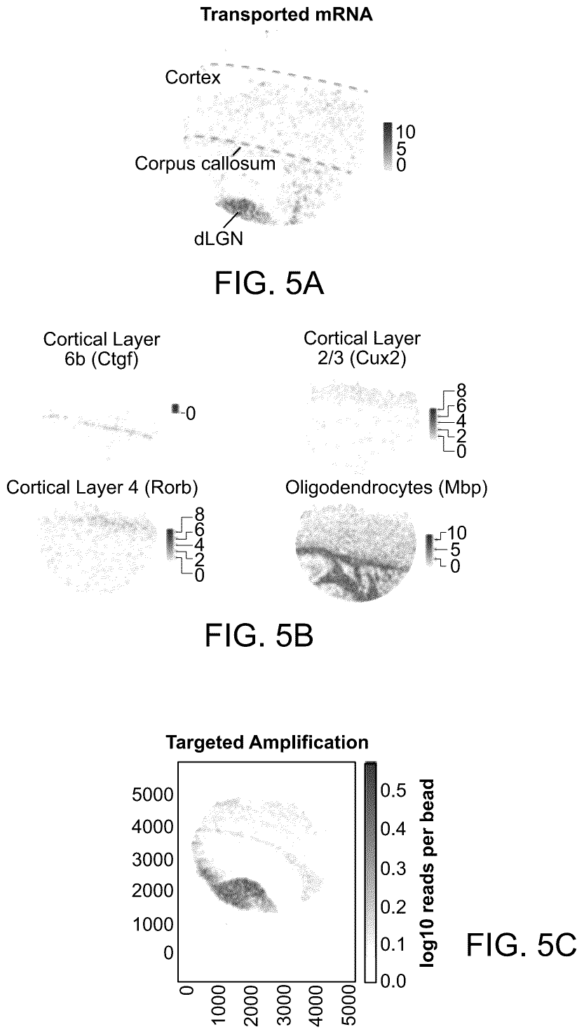

Therefore, in situ sequencing of AAV barcodes at synapses is performed. Specifically, the CA1 field is sectioned, and laser microdissection of the synaptic layers from the granular layer is performed. The granular layer and dissociate nuclei are collected and snRNA-seq is performed to ascertain a white list of AAV barcodes within CA1 pyramidal neurons. The synaptic layers are then formalin-fixed, and gapped padlock probes are used, targeting the AAV barcode transcript to fill in the barcode sequence, ligate, and generate rolling circle colonies (“rolonies”) in situ (FIG. 8).

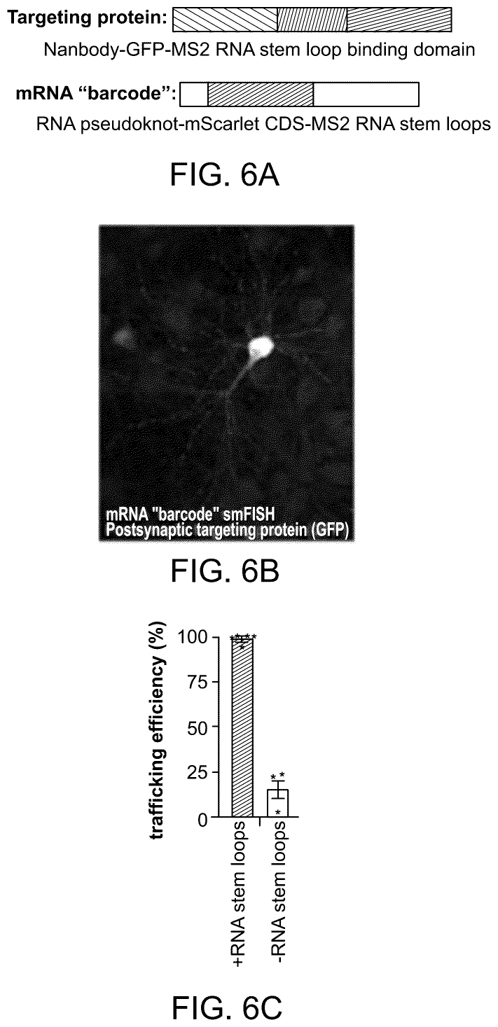

It was considered likely that an individual neuron could have been infected with multiple unique barcoded virions, and that there may be within-neuron diversity in the barcoding. To assess this prospect, protocols were developed for gap-fill ligation and in situ sequencing of resulting “rolony” (rolling circle colony) products. Crucially, it was observed that rolony amplification demonstrated spatial competition, in which only a single rolony could be amplified within a 200-500 nm sphere (size of rolony, FIG. 9). Therefore, regardless of the AAV expression rate, or number of unique barcodes per cell, which may be difficult to control with the AAV infectivity, it was expected that significant crowding problems that prevent the sequencing of individual rolonies could be avoided. Previously, 12 sequential bases in situ from genome products were sequenced (FIG. 9), which demonstrated the ability to resolve sufficiently diverse sequences and the ability to detect the instant barcoded AAV in vitro.

### Example 6: Proximity Detection In Situ for Identification of Paired Pre- and Postsynaptic Partners at Scale

Leveraging the pre- and postsynaptic barcoding systems was proposed for measuring neuronal projection patterns and synaptic spine density. The data herein shows the ability to label pre- and postsynaptic constructs with mRNA, and to barcode these mRNAs with virion-specific sequences, showing that utilization of the constructs disclosed herein could be used to develop readouts of cellular connectivity. Additional development of two alternative readouts of joint pre- and postsynaptic proximity is contemplated, as is validation therein in a well-established circuit in vivo.

Detection of pre- and post-synaptic barcode partners by polony network in situ (PONI) amplification is also contemplated. Such technologies enable the precise in situ colocalization of biomolecules at synapses—at resolutions exceeding the diffraction limits of microscopy. Several groups previously proposed to use in situ PCR amplification with local concatenation to detect spatial proximity between two nucleic acids. However, the instant disclosure is believed to provide a more viable means of providing spatial proximity data across a wide range of transcripts at a given subcellular location. Such methods for in situ molecular colocalization are enormously biologically enabling, and are especially useful for detecting synaptic connections, since individual synapses cannot be resolved by standard light microscopy.

Previous work proposed using in situ PCR as a potential means of detecting proximity interactions between biomolecules. In this strategy, diffusion is hard to control, since biomolecules are not tethered; furthermore, spatial reconstruction in silico is a highly non-convex optimization problem. POlony Network In situ sequencing (PONI-seq), a flexible, generalizable technology for measuring molecular interactions within tissue sections was contemplated and was previously described in PCT/U22/16144.

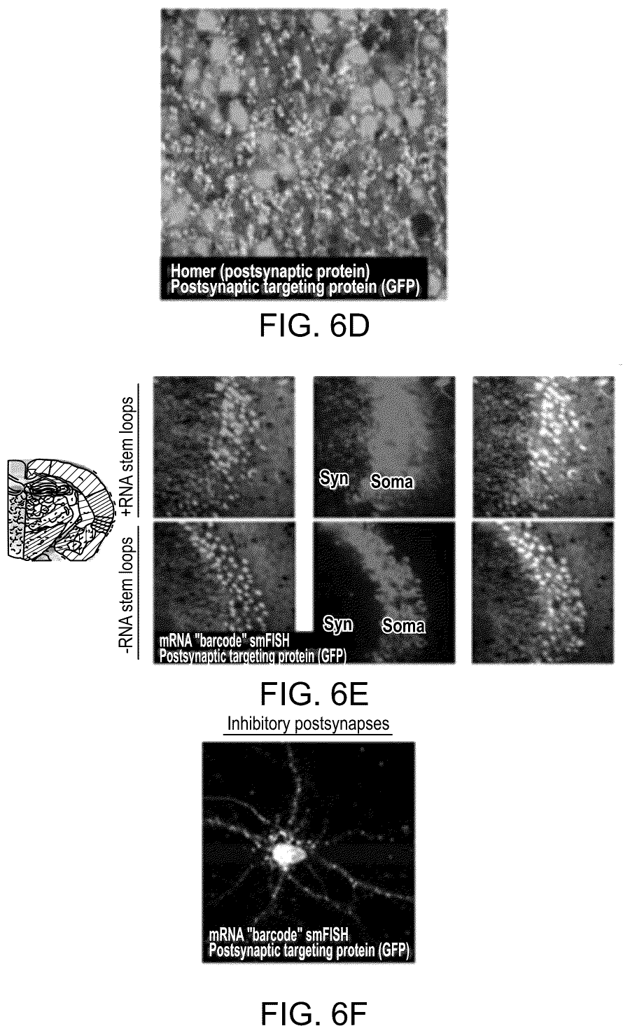

In PONI-seq, fixed tissue sections are first probed for biomolecules of interest with nucleic acid-tagged probes. These probes may be, for example, tagged primers for reverse transcription (to target RNA), or oligonucleotide-tagged antibodies (to detect proteins). A polymerization reaction incorporates modified oligonucleotides into a polymer network embedded within the fixed tissue section (FIG. 10). Crucially, the polymer network is not a gel, which has been found to inhibit downstream reactions, presumably by steric hindrance of hybridization or polymerization. The polymer-bound oligonucleotides are then used to prime a solid-phase, bridge amplification reaction in which a PCR product is expanded locally and remains tethered to the gel matrix, forming what is termed a “polony” in situ. As polonies grow and expand across bridge amplification cycles, they collide with each other, and can form concatemers (FIG. 10). Capture of the amplicons and concatemers on a Slide-seq array and subsequent high-throughput DNA sequencing enables the precise localization of polonies, inferred by the number of recombinant counts between amplified biomolecules. In this way, the number of recombination events can be computed between each pre- and postsynaptic barcode, generating an interaction matrix to digitally quantify synaptic connections.

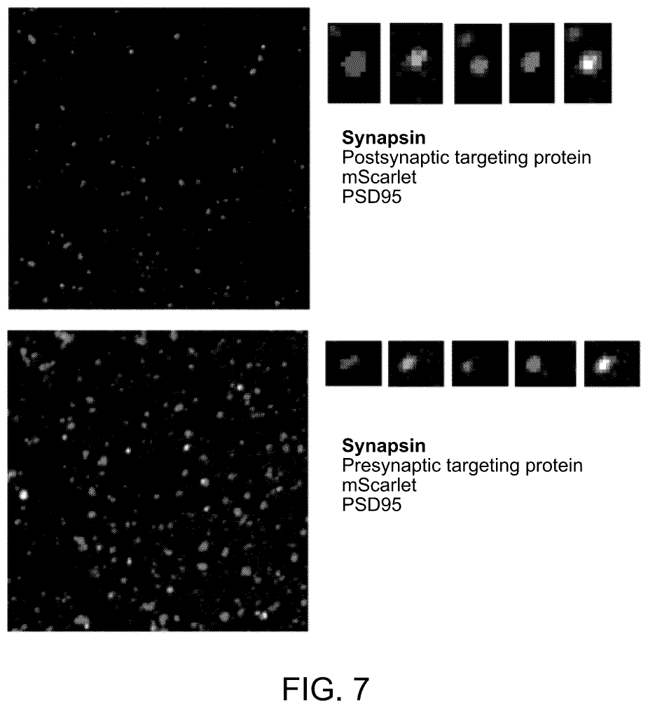

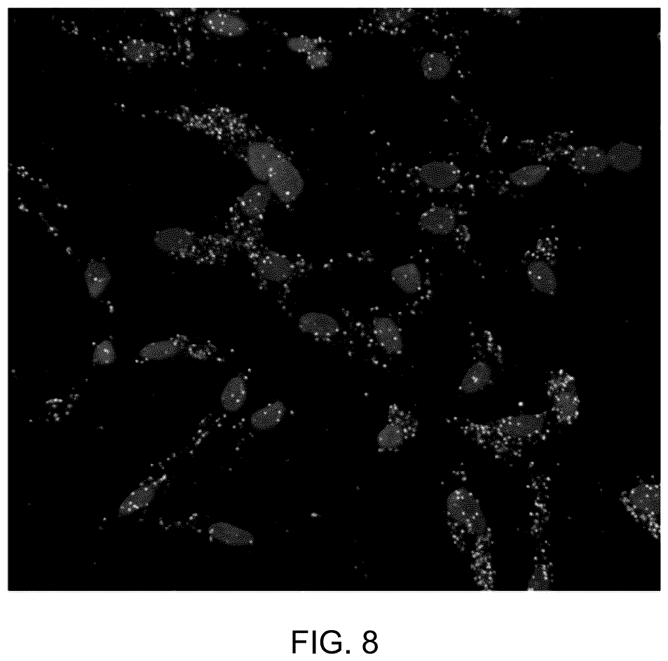

In exemplifying PONI-seq, reverse transcription was performed with a tagged oligo dT primer on mouse hippocampal tissue sections, the tissue was embedded with a lowly crosslinked polymer and polymer-bound primers targeting two genes with distinct spatial distributions: Mbp and Hpca. Bridge amplification was performed across different numbers of cycles to form polonies, and the polonies probed by single molecule fluorescence in situ hybridization (smFISH). The strength of the bridge amplification signal was dependent upon the presence of amplification primers and increased in intensity as the number of cycles of amplification was increased (FIG. 11). The smFISH signal was detected in the spatial distributions expected for each gene (FIG. 12), demonstrating that PONI amplification faithfully maintained the spatial distribution of biomolecules.

PONI-seq offers the capacity to combine the in situ detection of biomolecules with the quantification of molecular interactions between those molecules. For example, one group of antibodies is conjugated to an oligonucleotide that can template extension off of an oligonucleotide on a different group of antibodies. As PONIs expand during bridge amplification, neighboring amplicons collide and recombine, which can be detected by downstream DNA sequencing. The greater the number of detected proximity events, the closer the two original molecules are to each other in the tissue specimen.

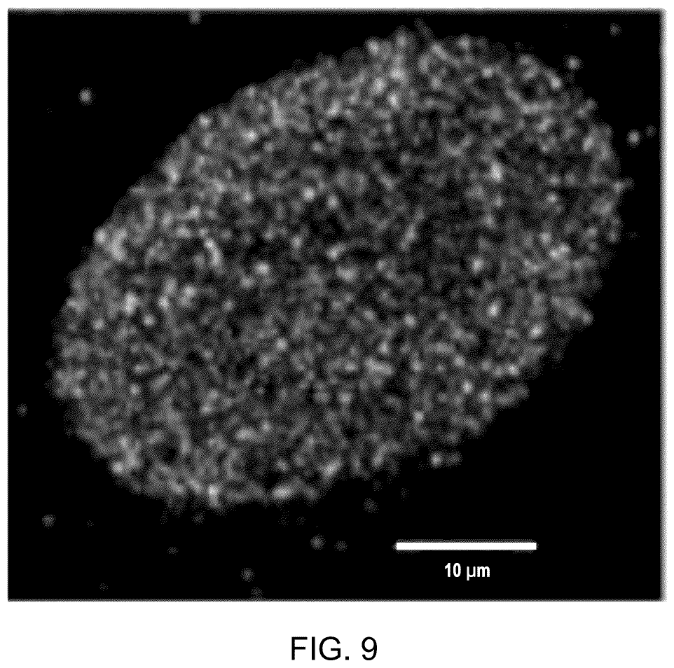

To explore the spatial resolution afforded by recombination, tissue was stained with oligonucleotide-tagged antibodies for RBFOX3 (a.k.a. NEUN, a neuronal marker) and OLIG2 (an oligodendrocyte marker) and PONI was performed on the whole transcriptome and these oligonucleotide tags. The genes with the highest recombination rate with each antibody were also strongly expressed in the respective cell type—for example, the neuron-specific gene Snap25 had the highest amount of recombination with RBFOX3, while the oligodendrocyte-specific gene Ptgds had the highest amount of recombination with OLIG2 (FIG. 13A). Next, antibody-RNA recombination was performed using antibodies targeting two nuclear proteins (RBFOX3 and H3) and two cytoplasmic proteins (SYN and GFAP). The percent of recombinant RNA reads mapping to intronic sequences was more than 2-fold higher for the two nuclear-localized proteins than the cytoplasmic proteins (FIG. 13B). These results demonstrated that PONI could report molecular proximity at subcellular resolution.

### Example 7: Validation and Generalization of an AAV-Delivered Postsynaptic Trafficking System In Vitro and In Vivo

To fully validate a postsynaptic trafficking system in vivo, gold-standard synaptic bouton counting experiments are employed, which are also expected to generalize the postsynaptic labeling to many different kinds of neurons in vivo. To simultaneously count dendritic spines by microscopy, and quantify trafficking of the mRNA in the same cells in vivo, a sparse, stochastic labeling system is implemented in which a FLEX switch toggles expression of the trafficked mRNA only in the presence of a second Cre-expressing virus, which is injected at low titer to only label tissue sparsely. The FLEX reporter also contains a soluble mScarlet reporter that fills the transduced neurons and allows the counting of dendritic spines by light microscopy. Simultaneously counting spines is performed (using immunohistochemistry), and quantifying mRNA labeling by smFISH, to generate a gold-standard assessment of the sensitivity and specificity of the construct in vivo. Moreover, this is compared with a FLEX reporter-only construct to confirm that the system does not impact spine number. This same sparse labeling system is used as a gold-standard validation in the barcode-based quantification of spines described herein.

Postsynaptic FingR has been applied to several neuronal cell types, but it is important to ensure that the FingR-based mRNA trafficking system is similarly generalizable. The sparse labeling approach is deployed to examine postsynaptic labeling in pyramidal neurons of prelimbic cortex, medium spiny neurons of the striatum, and Purkinje neurons in the cerebellum. This gold-standard validation approach is deployed to count spines in each of these neuronal populations, and compute sensitivity and specificity of labeling with the trafficked mRNA.

### Example 8: Generalization and Validation of AAV-Delivered Presynaptic Trafficking System In Vivo

Additional refinement and validation of the presynaptic targeting system is contemplated for the purpose of confirming that RNA is efficiently and specifically delivered to the presynaptic compartment, that endogenous presynaptic morphology or connectivity is maintained when system components of the disclosure are expressed, and to confirm that the current system is generally applicable to many cell types and circuits in the brain.

Improvements in specificity are also contemplated. In the in vivo experiment shown in FIG. 1C, some accumulation of mRNA outside of these boutons was observed, which indicated that greater specificity could be achieved. In vivo synaptic targeting proteins are therefore developed to generate a more specifically targeting presynaptic system, using three alternative approaches. First, a fusion of presynaptic targeting protein to a zinc finger-based transcriptional regulation system, which inhibits further transgene expression once trafficking sites are saturated, is employed with the current Synapse-seq system. This transcriptional control loop has been used to generate a highly specific postsynaptic targeting system, which could be useful for ensuring that imaging and detection levels remain quantitative, and to mitigate against potential toxicity of the Synapse-seq system, were expression to proceed in an unchecked manner. Secondly, the presynaptic targeting protein with the same zinc finger self-repressor is expressed using a P2A self-cleaving peptide. This is predicted to increase specificity by dampening expression levels of the targeting protein. Third, the optimization of both the number, location and type of RNA binding domains used in the present system to enhance RNA transport and binding is contemplated. Several alternatives to the MS2 RNA binding system exist, and previous studies have shown that RNA binding can be enhanced by tandem dimerization. In all cases, quantification is performed on the mRNA presynaptic labeling efficiency (number of presynapses labeled) and specificity (ratio of presynaptic fluorescence to total overall fluorescence) in vitro (using primary cortical neurons) and in vivo (using the hippocampal to RSP projection in FIG. 1C).

### Example 9: Generalization to Other Circuits

Projection neurons derive from different embryonic lineages, send their axons across vastly different length scales, and often utilize different molecular machinery for axonal trafficking and presynaptic release. For these reasons, it is important to assess the generality of the presynaptic barcoding construct by quantifying the efficiency of presynaptic mRNA labeling in three additional circuits: 1) thalamocortical projections from the VPM thalamic nucleus to frontal cortex; 2) dopaminergic projections from the ventral tegmental area to the nucleus accumbens; and 3) corticostriatal projections. For each of these three circuits, proper stereotactic injection targeting is confirmed by imaging tissue for AAV expression at the injection site, and then dual smFISH and presynaptic immunohistochemistry (using established markers such as synapsin) is performed on the trafficked mRNA at each target site, thereby quantifying presynaptic labeling efficiency and specificity.

Further validation of presynaptic labeling is also contemplated. Ectopic expression of presynaptic-binding components using a viral vector could have unanticipated effects on axonal health, morphology, and connectivity. To measure axonal morphology and presynaptic targeting simultaneously in the same cells in vivo, a sparse, stochastic labeling system based on these targeting constructs is employed, in which a FLEX switch toggles expression of the trafficked mRNA only in the presence of Cre, which is injected at low titer to label tissue only sparsely. Injection into CA3 is performed, labeling projections to RSP as shown in FIG. 1. Whole brain tissue clearing is utilized to visualize axonal morphology-specifically quantifying branching, layer targeting, and presynaptic bouton density—in the cells containing the targeting construct. The anatomy of these cells is compared to those sparsely labelled with just a FLEX GFP reporter. In addition to CA3, the same experiment is performed but with the present system injected into thalamocortical projections to the cortex. These experiments are expected to confirm fully that the targeting construct of the instant disclosure does not affect axonal health or morphology.

### Example 10: Other Contemplated Validations and Applications

AAV viral engineering is also used to develop a viral production protocol that maximizes viral barcode diversity and titer. To maximize the acquisition of barcodes from individual neuronal transcriptomes, targeted approaches are utilized to amplify and sequence mRNA reporter barcodes in parallel, either using specific amplification on cDNA libraries (see biotin-based target amplification strategy described in Example 1) or using an alternative capture sequence to polyA that is commercially available (from 10× Genomics). To evaluate these different approaches, the comparison of the median number of transcripts per unique barcode in each neuron is utilized.

Systematic, large-scale mapping of thalamocortical projections using Slide-seq as a readout is also performed. In the mammalian brain, projection neuron targeting is often highly spatially stereotyped. For example, thalamocortical neurons mostly target pyramidal neurons in layer 4, but additional connectivity—for example in layer 1—plays a very different role in circuit modulation. Developing a high-throughput tool that provides simultaneous cell-specific transcriptional characterization of projection neurons with spatial localization of projections is highly enabling for circuit neuroscience research.

To demonstrate the ability to quantify presynaptic projections with the present system, the VPM nucleus of the thalamus, which sends projections to somatosensory cortex, is injected with the presynaptically targeting AAV system of the instant disclosure. snRNA-seq is performed on the VPM to obtain a whitelist of AAV barcodes in specific VPM neurons, as well as to measure the transcriptional profiles of these projection neurons. On the same animal, Slide-seq is performed on 40 serial sagittal sections of somatosensory cortex, reconstructing approximately 20% of the regional volume. To map projections, the barcodes sequenced by Slide-seq is intersected with those sequenced in snRNA-seq, to digitally quantify the number of reconstructed cells.

This allows the sequencing of the synaptic barcode space in a highly efficient manner, enabling scaled experiments across multiple sections and brains. To understand how much AAV expression is needed to be presynaptically transported and detected with this approach, single nucleus RNA sequencing is performed on the injection site (thalamus), coupled with bulk RNA sequencing on the presynaptic projection site (cortex). Barcodes are read out with targeted amplification and sequencing, enabling the linkage of the expression level of barcodes in individual neurons to presynaptic trafficking. Critically, because of the high diversity of the AAV transcript (FIG. 4), and the high MOI of the projection neurons, only a small fraction of a cell's barcodes need to be matched between snRNA-seq and projection Slide-seq in order for it to be mapped.

A key test of the system is the detection of a perturbation that alters the total number of presynaptic boutons. To do this, Slide-seq is performed on the thalamocortical projections from the VPM, which transmit somatosensory information from the thalamus to the cortex. Abolishment of somatosensation during postnatal development (by lesioning of the whiskers at postnatal day 4) is known to produce extensive reduction of VPM projection neuron presynaptic number in the cortex without impacting the health of the VPM projection neurons. In addition, whisker lesioning can be performed unilaterally, enabling an internal control. The trafficking of the construct is quantified, and resulting changes due to whisker shaving, with smFISH first for quality control of trafficking performance. Slide-seq is then used to trace the projection patterns of individual VPM projection neurons, using snRNA-seq of the VPM nucleus to generate a white list of barcodes for matching to the Slide-seq data generated from somatosensory cortex. Validation of the sensitivity of Synapse-seq by quantifying changes in presynaptic punctae in the whisker shaving model is expected, as is the likelihood of this process to provide insight into the localization of remaining presynapses and the transcriptomic and spatial correlates of reduced cortical input. Analytically exploring which transcriptional signatures in VPM neurons correlate with presynaptic loss in the cortex is also contemplated.

The current system is also used to examine the SLM and SR layers of CA1, computing an absolute count of synapses in each cell. snRNA-seq data is used to relate each barcoded synapse back to a cell-of-origin within the CA1 granular layer, allowing for generation of a spatially resolved map of synaptic densities for each transcriptomically profiled cell in the region.

Validation of each readout can be performed using a sparse labeling system that allows comparison of the results of each of the three readout methods to ground-truth data obtained by counting dendritic spines. For the bulk sequencing and synaptosome-based readouts, the distribution of spine counts per cell determined by these approaches is compared with the distribution of counts per cell quantified by direct microscopy. For the in situ sequencing, the current targeting system can be delivered without sparse labeling into a Thy l reporter mouse. In these transgenic mice, identify individual cells that co-express the Thy l reporter together with the instant postsynaptic labeling system are identified. In these cells, spines are counted by light microscopy, and then spines are counted again by in situ sequencing of the postsynaptic targeting construct. This experiment is expected to prove that barcode sequencing can accurately count dendritic spines.

To calibrate the size scales of spatial proximity events that PONI-seq can detect to synapse scale, mouse brain tissue is stained with antibodies for three proteins: (1) BASSOON, a presynaptic protein; (2) PSD95 (Postsynaptic Density Protein 95, also known as DLG4 or Discs Large MAGUK Scaffold Protein 4), a postsynaptic protein found at excitatory synapses; and (3) GPHN (Gephyrin), a postsynaptic protein found only at inhibitory synapses. The PONI detection process is then performed for different numbers of cycles. If recombination occurs only between BASSOON and itself, and not with a postsynaptic protein, then amplification likely only occurs at a scale of hundreds of nanometers (since a synapse is about a cubic micron in volume). If BASSOON is able to recombine with GPHN and PSD95, but GPHN and PSD95 do not recombine with each other, then amplification and recombination likely spanned a few cubic microns. If all proteins recombine with each other, then PONI amplification is deemed to have been too widespread. In this way, the PONI process is both validated and optimizes for detection of interactions at the length scale of an individual synapse.

Upon validation and optimization of PONI recombination for synapse detection, the technology is applied to the current CA1 model circuit. Specifically, distinct presynaptic barcoded AAVs are injected into the entorhinal cortex and into the CA3 field, and a postsynaptic barcoded AAV is injected into the CA1 field. Laser microdissection of the SLM and SR synaptic layers from the granular layer is performed, and snRNA-seq is performed on the CA1 granular layer, as well as the two presynaptic inputs (entorhinal cortex and CA3), thereby obtaining white lists of presynaptic and postsynaptic barcodes. The PONI process is then performed upon the isolated SLM and SR layers. Based upon the known connectivity of CA1, synaptic connectivity (a) between the CA1 and entorhinal cortex in the SLM and (b) between CA1 and CA3 in the SR is expected to be established. In addition, the microcircuitry of the frontal cortex is also quantified, where it is expected that VIP interneurons are found to synapse onto SST and PV expressing neurons, while PV and SST are expected to be measured as presynaptic to pyramidal neurons. Importantly, the current barcode structures and sequencing primers for the pre- and postsynaptic constructs are different, allowing for easy resolution of pre- and postsynaptic pairs at the start of the sequencing experiment.

To effectively tune PONI, introduction of a 3′ blocked RNA “cap” can also be added to PONI amplification oligos. Since these caps can only be removed by the addition of RNase H, iteration can be performed between amplification cycles with and without RNase H, maximizing recombination at different length scales. It is also possible that the amount of PONI amplification, and therefore the distance of diffusion, may vary between each experiment. Therefore, for each experiment, the tissue can be stained with control antibodies (such as the ones used for the validation and optimization of PONI-BASSOON, PSD95, and GPHN), to serve as internal controls or measures of PONI amplification.

Pre- and post-synaptic barcode partners can be detected by in situ sequencing. Protocols have been developed for generating gap-filled rolonies (rolling circle colonies) from tissue sections and sequencing up to 12 bases of the native transcript. Crucially, these rolonies display volume-filling competition, such that only a single rolony is amplified within a ˜200 nm sphere, reducing the problem of barcode overlap when trying to sequence densely infected neurons. This advantage is leveraged to jointly sequence both the pre- and postsynaptic barcoding systems, together with an in situ sequencing readout, to identify individual synaptic boutons formed between cells.

Individual pairs of connected neurons are rare in the hippocampus, and are expected to form multiple synapses with each other, based on available data. It is therefore expected that the current individually sequenced synaptic barcode pairs will follow the same distribution, with the same cell pair having multiple barcode pairs sequenced in the same experiment. If this distribution is not observed experimentally, this would indicate that current microscopy resolution is insufficient to resolve individual synapses. Two alternative strategies are contemplated to address this problem. First, use of expansion microscopy, in which tissue is embedded in an expandable hydrogel in order to resolve extremely small structures, like synapses, can be employed. In situ sequencing can also be performed with high efficiency in expanded tissues. Second, reading out the rolony proximity by sequencing, rather than by light microscopy, is attempted. In this approach, individual rolonies are either bridge ligated or digested and overlap extended. The rolony barcode pairs are then captured on a Slide-seq array and sequenced, similar to the capture of PONI recombinants. The rate of rolony detection by Slide-seq is extremely high-greater than 50% efficient and on the order of smFISH—making it an appealing read-out framework for synapses, which require pairwise detection.

Although synaptic connectivity forms the basis of brain function, and is implicated in a large number of CNS diseases, available tools to measure it are difficult to scale, and hard to combine with molecular definitions of neuronal cell types. The current system, Synapse-seq, combines innovations in the trafficking of barcoded mRNA to synaptic compartments with new and existing moleculo-spatial assays to enable the single-cell measurement of neuron projections, dendritic spine density, and cell-type-specific synaptic connectivity. The present disclosure therefore provides a new suite of tools for understanding the relationship between nervous system structure and function, with both research and clinical implications.

All patents and publications mentioned in the specification are indicative of the levels of skill of those skilled in the art to which the disclosure pertains. All references cited in this disclosure are incorporated by reference to the same extent as if each reference had been incorporated by reference in its entirety individually.

One skilled in the art would readily appreciate that the present disclosure is well adapted to carry out the objects and obtain the ends and advantages mentioned, as well as those inherent therein. The methods and compositions described herein as presently representative of preferred embodiments are exemplary and are not intended as limitations on the scope of the disclosure. Changes therein and other uses will occur to those skilled in the art, which are encompassed within the spirit of the disclosure, are defined by the scope of the claims.

In addition, where features or aspects of the disclosure are described in terms of Markush groups or other grouping of alternatives, those skilled in the art will recognize that the disclosure is also thereby described in terms of any individual member or subgroup of members of the Markush group or other group.

The use of the terms “a” and “an” and “the” and similar referents in the context of describing the disclosure (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein.

All methods described herein can be performed in any suitable order unless otherwise indicated herein or otherwise clearly contradicted by context. The use of any and all examples, or exemplary language (e.g., “such as”) provided herein, is intended merely to better illuminate the disclosure and does not pose a limitation on the scope of the disclosure unless otherwise claimed. No language in the specification should be construed as indicating any non-claimed element as essential to the practice of the disclosure.

Embodiments of this disclosure are described herein, including the best mode known to the inventors for carrying out the disclosed invention. Variations of those embodiments may become apparent to those of ordinary skill in the art upon reading the foregoing description.

The disclosure illustratively described herein suitably can be practiced in the absence of any element or elements, limitation or limitations that are not specifically disclosed herein. Thus, for example, in each instance herein any of the terms “comprising”, “consisting essentially of”, and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present disclosure provides preferred embodiments, optional features, modification and variation of the concepts herein disclosed may be resorted to by those skilled in the art, and that such modifications and variations are considered to be within the scope of this disclosure as defined by the description and the appended claims.

It will be readily apparent to one skilled in the art that varying substitutions and modifications can be made to the invention disclosed herein without departing from the scope and spirit of the invention. Thus, such additional embodiments are within the scope of the present disclosure and the following claims. The present disclosure teaches one skilled in the art to test various combinations and/or substitutions of chemical modifications described herein toward generating conjugates possessing improved contrast, diagnostic and/or imaging activity. Therefore, the specific embodiments described herein are not limiting and one skilled in the art can readily appreciate that specific combinations of the modifications described herein can be tested without undue experimentation toward identifying conjugates possessing improved contrast, diagnostic and/or imaging activity.

The inventors expect skilled artisans to employ such variations as appropriate, and the inventors intend for the disclosure to be practiced otherwise than as specifically described herein. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context. Those skilled in the art will recognize, or be able to ascertain using no more than routine experimentation, many equivalents to the specific embodiments of the disclosure described herein. Such equivalents are intended to be encompassed by the following claims.

1. A composition for tagging the localization of a fusion protein in a cell, tissue or organism, the composition comprising:
a) a first plasmid capable of being expressed in a cell, wherein the first plasmid encodes for the fusion protein, wherein the fusion protein comprises a first domain comprising a vesicle-, synapse- and/or organelle-associated protein or a polypeptide sequence that binds a vesicle-, synapse- and/or organelle-associated protein and a second domain comprising a selective nucleic acid binding protein; and
b) i) a second plasmid capable of being expressed in a cell, wherein the second plasmid encodes for an oligoribonucleotide comprising a selective protein binding nucleic acid domain, and a barcode nucleic acid, wherein the selective protein binding nucleic acid domain is capable of binding the selective nucleic acid binding protein of the first plasmid; or
ii) an oligonucleotide comprising a selective protein binding nucleic acid domain, and a barcode nucleic acid, wherein the selective protein binding nucleic acid domain is capable of binding the selective nucleic acid binding protein of the first plasmid.

2. The composition of claim 1, wherein the selective nucleic acid binding protein is a selective RNA binding protein and the selective protein binding nucleic acid domain is a selective protein binding RNA domain.

3. The composition of claim 1, wherein the vesicle-, synapse- and/or organelle-associated protein or the polypeptide sequence that binds the vesicle-, synapse- and/or organelle-associated protein is selected from the group consisting of a synaptic vesicle marker, a presynaptic synapse marker, a postsynaptic synapse marker, a ribosomal marker, a gap junction marker, a lysosomal marker, and an endosomal marker.

4. The composition of claim 1, wherein the selective nucleic acid binding protein and the selective protein binding nucleic acid domain comprise a zinc finger-based transcriptional regulation system.

5-6. (canceled)

7. The composition of claim 1, wherein:
the cell, tissue or organism is a mammalian cell, tissue or organism,
the selective RNA binding protein and the selective protein binding nucleic acid domain comprise a pair selected from the group consisting of: an MS2 coat protein (MCP) and an MS2 phage operator stem-loop, an RNA-binding section of the MCP and an MS2 phage operator stem-loop, a PP7 coat protein (PCP) and a PP7 phage operator stem-loop, an RNA-binding section of the PCP and a PP7 phage operator stem-loop, a Ku protein and a telomerase Ku binding motif, an RNA-binding section of the Ku protein and a telomerase Ku binding motif, an Sm7 protein and a telomerase Sm7 binding motif, an RNA-binding section of the Sm7 protein and a telomerase Sm7 binding motif, a Com RNA binding protein and a SfMu phage Com stem-loop, an RNA-binding section of the Com RNA binding protein and a SfMu phage Com stem-loop, an aptamer ligand and a corresponding non-natural RNA aptamer, and an RNA-binding section of an aptamer ligand and a corresponding non-natural RNA aptamer,
the selective RNA binding protein comprises a MCP and the selective protein binding RNA domain comprises a MS2 phage operator stem-loop, or the selective RNA binding protein comprises a PP7 coat protein (PCP) and the selective protein binding RNA domain comprises a PP7 phage operator stem-loop,
the vesicle-, synapse- and/or organelle-associated protein or the polypeptide sequence that binds the vesicle-, synapse- and/or organelle-associated protein is selected from the group consisting of a protein comprising a synaptophysin domain, a protein comprising a fibronectin intrabody, an α-synuclein-binding FingR, a Bassoon-binding FingR, a PSD95-binding FingR, and a GPHN-binding FingR,
the mammalian cell is a neuron, and/or
the mammalian cell is a neuron in vivo.

8-10. (canceled)

11. A mammalian cell comprising the composition of claim 1.

12. A virus comprising the composition of claim 1, wherein the virus is a non-toxic virus for infection of mammalian cells.

13. A method for detecting the localization of a fusion protein in a cell, tissue or organism, the method comprising:
a) administering the composition of claim 1 to the cell, tissue or organism;
b) providing conditions suitable for fusion protein expression, binding of the selective protein binding nucleic acid domain to the selective nucleic acid binding protein, and time sufficient for localization of the bound selective protein binding nucleic acid domain in the cell, tissue or organism to occur; and
c) applying a spatially-localized sequencing assay or platform to at least a portion of the cell, tissue or organism, thereby obtaining sufficient sequence and location information to detect the localization of a barcode sequence within the cell, tissue or organism,
thereby detecting the localization of the fusion protein in the cell, tissue or organism.

14. The method of claim 13, wherein the spatially-localized sequencing assay or platform comprises obtaining a tissue section of the cell, tissue or organism and contacting the tissue section with a tagged array that retains sequence information while next generation sequencing (NGS) is performed.

15. The method of claim 13 further comprising obtaining single-cell sequence from the cell, tissue, or organism wherein the single-cell sequence obtains sequence of an injection site.

16. The method of claim 13, wherein applying the spatially-localized sequencing assay or platform comprises contacting the cell, tissue or organism with a first monomer or linear polymer and a cross-linking agent comprising a second monomer or polymer, wherein the cross-linking agent is capable of crosslinking with the first monomer or linear polymer when combined.

17. (canceled)

18. The method of claim 13, wherein the cell, tissue or organism is contacted with a gapped padlock probe, wherein the gapped padlock probe targets a barcode transcript to fill in the barcode sequence, further comprising ligating the gapped padlock probe comprising the barcode sequence and generating rolling circle colonies in situ.

19-21. (canceled)

22. The method of claim 13, wherein the spatially-localized sequencing assay or platform is applied to a pre-synaptic neuron, a post-synaptic neuron, an excitatory post-synaptic neuron, a cell that forms a chemical synapse, a cell that forms an electrical synapse, a cell that forms a gap junction, or a cell that form a δ-Notch immune synapse.

23-27. (canceled)

28. The method of claim 13, wherein the spatially-localized sequencing assay or platform comprises a quantitative spatial oligonucleotide sequencing system.

29. The method of claim 13, wherein the barcode sequence is detected with spatial resolution of about 10 μm or less.

30. The method of claim 13, further comprising determining spatial proximity of two or more barcode sequences by measuring the frequency of recombination events between amplicons of the two or more barcode sequences.

31. (canceled)

32. A method for delivering a barcode nucleic acid to a subcellular compartment or organelle of a mammalian cell, the method comprising contacting the mammalian cell with: a) a first plasmid capable of being expressed in the cell, wherein the first plasmid encodes for a fusion protein, wherein the fusion protein comprises a first domain comprising a subcellular compartment and/or organelle-associated protein or a polypeptide sequence that binds a subcellular compartment and/or organelle-associated protein and a second domain comprising a selective nucleic acid binding protein; and b) i) a second plasmid capable of being expressed in the cell, wherein the second plasmid encodes for an oligoribonucleotide comprising a selective protein binding nucleic acid domain, and a barcode nucleic acid, wherein the selective protein binding nucleic acid domain is capable of binding the selective nucleic acid binding protein of the first plasmid; or ii) an oligonucleotide comprising a selective protein binding nucleic acid domain, and a barcode nucleic acid, wherein the selective protein binding nucleic acid domain is capable of binding the selective nucleic acid binding protein of the first plasmid; under suitable conditions for intracellular trafficking and localization to occur,
thereby delivering the barcode nucleic acid to a subcellular compartment or organelle of the mammalian cell.

33. The method of claim 32, wherein;
the subcellular compartment is selected from the group consisting of a synaptic vesicle, a presynaptic synapse, a postsynaptic synapse, a ribosome, a gap junction, a lysosome, and an endosome, and/or the suitable conditions for intracellular trafficking and localization comprise: binding of the selective protein binding nucleic acid domain to the selective nucleic binding protein and time sufficient for localization of the bound selective protein binding nucleic acid domain in the subcellular compartment or organelle of the mammalian cell.

34. (canceled)

35. The composition of claim 4, wherein:
the zinc finger-based transcriptional regulation system is capable of inhibiting further transgene expression once trafficking sites for the fusion protein are saturated, and/or
the zinc finger-based transcriptional regulation system comprises an MS2 binding protein.

36. A system for determining the localization of a fusion protein in a cell of a subject, the system comprising:
a first plasmid and a second plasmid, wherein the first plasmid encodes for the fusion protein, wherein the fusion protein comprises a first domain comprising a trafficking protein and a second domain comprising a selective RNA binding protein, and wherein the second plasmid encodes for an oligoribonucleotide comprising a selective protein binding RNA domain and a barcode nucleic acid, wherein the selective protein binding RNA domain is capable of binding the selective RNA binding protein of the first plasmid;
packaging the first plasmid and the second plasmid in a virus;
introducing the virus to the subject, wherein upon cellular expression of the fusion protein of the first plasmid and cellular expression of the oligoribonucleotide of the second plasmid, the selective RNA binding protein binds the selective protein binding RNA domain and is thereby trafficked within the cell; and
detecting the barcode nucleic acid;
thereby determining the localization of the fusion protein in the cell of the subject.
